(19) United States
(12) Patent Application Publication
(10) Pub. No.: US 2003/0232742 A1
(43) Pub. Date: Dec. 18, 2003

(54) FAP-activated anti-tumor compounds

(76) Inventors: Stefan Peters, Biberach (DE); Dietmar Leipert, Rheinfelden (DE); Christian Eickmeier, Danbury, CT (US); John Edward Park, Biberach (DE); Martin Lenter, Ulm (DE); Pilar Garin-Chesa, Biberach (DE); Juergen Mack, Biberach (DE)

Correspondence Address: BOEHRINGER INGELHEIM CORPORATION, 900 RIDGEBURY ROAD, P. O. BOX 368, RIDGEFIELD, CT, 06877, US

(21) Appl. No.: 10/036,224

(22) Filed: Nov. 9, 2001

Related U.S. Application Data
(60) Provisional application No. 60/262,323, filed on Jan. 17, 2001.

(30) Foreign Application Priority Data
Nov. 10, 2000 (GB) 0027552.9

(51) Int. Cl. A61K 38/00; A61K 31/7008; A61K 31/54; A61K 31/537; A61K 31/496; A61K 31/454; A61K 31/426; A61K 31/421; A61K 31/401; A61K 31/4172; A61K 31/397

(52) U.S. Cl. 514/2; 514/114; 514/210.17; 514/227.5; 514/237.8; 514/252.12; 514/317; 514/365; 514/374; 514/400; 514/408; 530/403; 536/53; 544/59; 544/157; 544/159; 544/400; 546/22; 546/235; 548/204; 548/236; 548/338.1; 548/950

(57) ABSTRACT

The invention relates to a prodrug that is capable of being converted into a drug by the catalytic action of human fibroblast activation protein (FAPα), said prodrug is chemically stable under physiological conditions and can be used for the manufacture of physically stable aqueous formulations. It has a cleavage site which is recognised by FAPα, and the drug released by the enzymatic activity of FAPα is cytotoxic or cytostatic under physiological conditions.

### RELATED APPLICATIONS

The benefit of priority U.S. Provisional application No. 60/262,323, filed Jan. 17, 2001 is hereby claimed.

### FIELD OF THE INVENTION

The present invention relates to the field of tumor treatment by administration of a prodrug that is converted into a drug at the site of the tumor. In particular, the invention relates to prodrugs which may be converted into a drug by the catalytic action of FAPα, their manufacture and pharmaceutical use.

### BACKGROUND AND PRIOR ART

The human fibroblast activation protein (FAPα) is a Mᵣ 95,000 cell surface molecule originally identified with monoclonal antibody (mAb) F19 (Rettig et al. (1988) Proc. Natl. Acad. Sci. USA 85, 3110-3114; Rettig et al. (1993) Cancer Res. 53, 3327-3335). The FAPα cDNA codes for a type II integral membrane protein with a large extracellular domain, trans-membrane segment, and short cytoplasmic tail (Scanlan et al. (1994) Proc. Natl. Acad. Sci. USA 91, 5657-5661; WO 97/34927). FAPα shows 48% amino acid sequence identity to the T-cell activation antigen CD26, also known as dipeptidyl peptidase IV (DPPIV; EC 3.4.14.5), a membrane-bound protein with dipeptidyl peptidase activity (Scanlan et al., loc. cit.). FAPα has enzymatic activity and is a member of the serine protease family, with serine 624 being critical for enzymatic function (WO 97/34927). Work using a membrane overlay assay revealed that FAPα dimers are able to cleave Ala-Pro-7-amino-4-trifluoromethyl coumarin, Gly-Pro-7-amino-4-trifluoromethyl coumarin, and Lys-Pro-7-amino-4-trifluoromethyl coumarin dipeptides (WO 97/34927).

FAPα is selectively expressed in reactive stromal fibroblasts of many histological types of human epithelial cancers, granulation tissue of healing wounds, and malignant cells of certain bone and soft tissue sarcomas. Normal adult tissues are generally devoid of detectable FAPα, but some foetal mesenchymal tissues transiently express the molecule. In contrast, most of the common types of epithelial cancers, including >90% of breast, non-small-cell lung, and colorectal carcinomas, contain FAPα-reactive stromal fibroblasts (Scanlan et al., loc. cit.). These FAPα⁺ fibroblasts accompany newly formed tumor blood vessels, forming a distinct cellular compartment interposed between the tumor capillary endothelium and the basal aspect of malignant epithelial cell clusters (Welt et al. (1994) J. Clin. Oncol. 12(6), 1193-1203). While FAPα⁺ stromal fibroblasts are found in both primary and metastatic carcinomas, the benign and premalignant epithelial lesions tested (Welt et al., loc. cit.), such as fibroadenomas of the breast and colorectal adenomas, only rarely contain FAPα⁺ stromal cells. Based on the restricted distribution pattern of FAPα in normal tissues and its uniform expression in the supporting stroma of many malignant tumors, clinical trials with ¹³¹I-labeled mAb F19 have been initiated in patients with metastatic colon carcinomas (Welt et al., loc. cit.).

For new cancer therapies based on cytotoxic or cytostatic drugs, a major consideration is to increase the therapeutic index by improving the efficacy of cancerous tissue killing and/or reducing the toxicity for normal tissue of the cytotoxic or cytostatic agents. To increase specificity of tumor tissue killing and reduce toxicity in normal tissues, trigger mechanisms can be designed so that the toxic agents synthesised in their prodrug or inactive forms are rendered active when and where required, notably in the cancerous tissues (Panchal (1998) Biochem. Pharmacol. 55, 247-252). Triggering mechanisms may include either exogenous factors such as light or chemicals or endogenous cellular factors, such as enzymes with restricted expression in cancer tissues. Another concept, that has been further elaborated, is called ‘antibody-directed enzyme prodrug therapy’ (ADEPT) or ‘antibody-directed catalysis’ (ADC) (Huennekens (1994) Trends Biotechnol 12, 234-239; Bagshawe (1994) Clin. Pharmacokinet. 27, 368-376; Wang et al. (1992) Cancer Res. 52, 4484-4491; Sperker et al. (1997) Clin. Pharmacokinet. 33(1), 18-31). In ADEPT, an antibody directed at a tumor-associated antigen is used to target a specific enzyme to the tumor site. The tumor-located enzyme converts a subsequently administered prodrug into an active cytotoxic agent. The antibody-enzyme conjugate (AEC) binds to a target antigen on cell membranes or to free antigen in extracellular fluid (ECF). A time interval between giving the AEC and prodrug allows for the AEC to be cleared from normal tissues so that the prodrug is not activated in the normal tissues or blood. However, some disadvantages of ADEPT are related to the properties of the AEC (Bagshawe, loc. cit.). For example, in humans, only a small fraction of the administered dose of the targeting AEC binds to tumor tissue and the remainder is distributed through body fluids from which it is cleared with significant time delays. Even very low concentrations of unbound enzyme can catalyse enough prodrug to have toxic effects because plasma and normal ECF volumes are much greater than those of tumor ECF. The AEC may also be immunogenic, thus preventing repeat administration, in many instances.

The International patent applications WO 97/12624 and WO 97/14416 disclose oligopeptides including the following penta- and hexapeptide (SEQ.ID.NOs.: 151 and 177: hArg-Tyr-Gln-Ser-Ser-Pro; hArg-Tyr-Gln-Ser-Pro;), comprising amino acid sequences, which are recognized and proteolytically cleaved by free prostate specific antigen (PSA) and therapeutic agents which comprise conjugates of such oligopeptides and known therapeutic or cytotoxic agents. These oligopeptide conjugates which comprise at least one glutamine-serine moiety are useful for treatment of prostate cancer only.

The problem underlying the present invention was to provide methods and means for improving normal tissue tolerability of cytotoxic or cytostatic agents with known efficacy against a broad range of tumor tissues, which can be administered to patients in need thereof in a safe and convenient way.

### DISCLOSURE OF THE INVENTION

The present invention relates to enzyme-activated anti-tumor compounds. In particular, the invention provides prodrugs which are capable of being converted into a cytotoxic or cytostatic drug, by the catalytic action of FAPα, said prodrugs exhibit an oligomeric part comprising up to 13 amino carboxylic residues, the C-terminal amino carboxylic thereof is recognised by FAPα, and a cytotoxic or cytostatic part, wherein the N-terminal amino function of the oligomeric part is attached to a capping group (Cg) which is capable of enhancing the chemical stability of said prodrug under physiological conditions and the physical stability of an aqueous pharmaceutical formulations comprising said prodrug.

In the context of this invention, a “drug” shall mean a chemical compound that may be administered to humans or animals as an aid in the treatment of disease. In particular, a drug is an active pharmacological agent.

The term “cytotoxic compound” shall mean a chemical compound which is toxic to living cells, in particular a drug that destroys or kills cells. The term “cytostatic compound” shall mean a compound that suppresses cell growth and multiplication and thus inhibits the proliferation of cells. Examples for cytotoxic or cytostatic compounds suitable for the present invention are anthracycline derivatives such as doxorubicin, analogs of methotrexate such as methothrexate, pritrexime, trimetrexate or DDMP, melphalan, analogs of cisplatin such as cisplatin, JM216, JM335, bis(platinum) or carboplatin, analogs of purines and pyrimidines such as cytarbine, gemcitabine, azacitidine, 6-thioguanine, flurdarabine or 2-deoxycoformycin, and analogs of other chemotherapeutic agents such as 9-aminocamptothecin, D,L-aminoglutethimide, trimethoprim, pyrimethamine, mitomycin C, mitoxantrone, cyclophosphanamide, 5-fluorouracil, extramustine, podophyllotoxin, bleomycin or taxol.

A “prodrug” shall mean a compound that, on administration, must undergo chemical conversion by metabolic processes before becoming an active pharmacological agent. In particular, a prodrug is a precursor of a drug. In the context of the present invention, the prodrug is significantly less cytotoxic or cytostatic than the drug it is converted into upon the catalytic action of FAPα. The expert knows methods of determining cytotoxicity of a compound, see e.g. example 45 herein, or Mosmann ((1983) J. Immun. Meth. 65, 55-63). Preferably, the prodrug is at least three times less cytotoxic as compared to the drug in an in vitro assay.

A “drug being cytostatic or cytotoxic under physiological conditions” shall mean a chemical compound which is cytostatic or cytotoxic in a living human or animal body, in particular a compound that kills cells or inhibits proliferation of cells within a living human or animal body.

A “prodrug having a cleavage site which is recognised by FAPα” shall mean a prodrug which can act as a substrate for the enzymatic activity of FAPα. In particular, the enzymatic activity of FAPα can catalyse cleavage of a covalent bond of the prodrug under physiological conditions. By cleavage of this covalent bond, the prodrug is converted into the drug, either directly or indirectly. Indirect activation would be the case if the cleavage product of the FAPα catalysed step is not the pharmacologically active agent itself but undergoes a further reaction step, e.g. hydrolysis, to become active. More preferably, the cleavage site of the prodrug is specifically recognised by FAPα, but not by other proteolytic enzymes present in the human or animal body. Also preferably, the cleavage site is specifically recognised by FAPα, but not by proteolytic enzymes present in human or animal body fluids, especially plasma. In a particularly preferred embodiment, the prodrug is stable in plasma, other body fluids, or tissues, in which biologically active FAPα is not present or detectable. Preferably, in an in vitro assay as carried out in Example 46 herein, more than 50%, more preferably more than 80%, more preferably more than 90% of the prodrug are still present in a solution containing 10% (v/v) of human plasma after 8 h at 37° C. The cleavage site should most preferably be specific for FAPα. In a preferred embodiment, the cleavage site comprises a L-proline residue which is linked to a cytotoxic or cytostatic drug via an amide bond. An example of this class is a doxorubicin-peptide conjugate. FAPα may catalyse the cleavage of a peptidic bond between the C-terminal amino acid residue of the peptide, which is preferably L-proline, and the cytotoxic or cytostatic compound.

Preferred compounds show at least 10% conversion to free drug, under standard conditions listed below. More preferred are compounds that show at least 20% conversion to free drug, under standard conditions. Even more preferred are compounds that show at least 50% conversion to free drug, under standard conditions. In this context, standard conditions are defined as follows: Each compound is dissolved in 50 mM Hepes buffer, 150 mM NaCl, pH 7.2, at a final concentration of 5 μM and incubated with 100 ng CD8FAPα (see example 43) for 24 hours at 37° C. Release of free drug by CD8FAPα is determined as described in example 44.

Preferably, the present invention relates to a compound of formula (I)

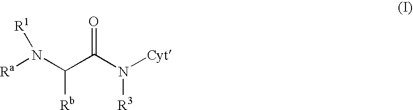

or a pharmaceutically acceptable salt thereof,

wherein

R¹ represents an amino alkanoyl or oligopeptidoyl group, the N-terminal amino function of which is attached to a capping group (Cg) which is capable of enhancing the chemical stability of said compound under physiological conditions and the physical stability of aqueous pharmaceutical formulations comprising said compound;

R^{a} and R^{b} together with the interjacent N—C group form an optionally substituted, optionally benzo- or cyclohexano-condensed 3- to 7-membered saturated or unsaturated heterocyclic ring, in which one or two CH₂ groups may also be replaced by NH, O or S;

R³ represents H, C₁-C₆-alkyl, C₃-C₈-cycloalkyl, aryl or heteroaryl; and

Cyt′ represents the residue of a cytotoxic or cytostatic compound.

Preferred are those compounds of formula I, wherein the capping group exhibits one or more functional groups, which have the capability of forming salts with pharmaceutially acceptable acids or bases, selected from amino, carboxy, phospate, phophonate, sulfate and sulfonate groups.

Particularly preferred are those compounds of formula I, wherein said capping group (Cg) is a group of formula

R²—(CH₂)ₘ-Z-,

in which

R² represents

(a) a group selected from C₁-C₆ alkyl, C₃-C₈ cycloalkyl, aryl and heteroaryl, wherein each of these groups is substituted by at least one amino, amino-C₁-C₆ alkyl, C₁-C₆ alkylamino, di-(C₁-C₆ alkyl)-amino, carboxy, phospate, phophonate, sulfate, sulfonate or hydroxy group, preferably selected from C₁-C₆ alkyl, phenyl thiazol and pyridine, wherein each of these groups is substituted by at least one amino, aminomethyl, methylamino, dimethylamino, carboxy, or hydroxy group,

(b) an optionally substituted 5- to 7-membered saturated or unsaturated nitrogen, oxygen and/or sulfur containing heterocyclic group, preferably a heterocyclic group selected from pyridine, pyrimidine, pyrazine, thiazol, pyrrol, imidazole, piperidine, piperzaine, N-methylpyrrolidine, morpholine and thiomorpholine,

(c) a phenyl group which is substituted by 1 to 5 fluorine atoms; preferably a phenyl group which is substituted by 1, 2 or 3 fluorine atoms,

(d) a C₁-C₆ fluoroalkyl group; or

(e) in the case that m is 1, an optionally substituted 5- to 6-membered heteroaryl group, preferably selected from pyridine and pyrimidine;

Z represents —CO—, —O—CO—, —SO₂—, NH—CO— or a single bond;

m is 0, 1 or 2, preferably 0 or 1.

Furthermore preferred are those compounds of formula I, wherein

R¹ represents a residue of formula Cg-A, Cg-B-A or Cg-(D)ₙ-B-A, in which Cg represents a capping group of formula R²—CH₂-Z-, wherein R² is an optionally substituted saturated heterocyclyl or heteroaryl group;

A, B and D each independently represent moieties derived from amino carboxylic acids of the formula —[NR⁴—(X)ₚ—CO]— wherein X represents CR⁵R⁶ and wherein R⁴, R⁵ and R⁶ each independently represent a hydrogen atom, an optionally substituted C₁-C₆-alkyl, C₃-C₈-cycloalkyl, aryl, aralkyl, heteroaryl or heteroarylalkyl group, and p is 1, 2, 3, 4, 5; or

A, B and D each independently represent moieties derived from cyclic amino carboxylic acids of formula

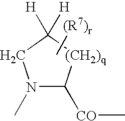

wherein

R⁷ represents C₁-C₆-alkyl, OH, or NH₂,

n is an integer from 1 to 10;

q is 0, 1 or 2; and

r is 0, 1 or 2, in particular wherein

R¹ represents an aminoalkanoyl, or an oligopeptidoyl group, which is derived from glycine (Gly), or the D- or L-forms, in particular the (L)-configuration of alanine (Ala), valine (Val), leucine (Leu), isoleucine (Ile), phenylalanine (Phe), tyrosine (Tyr), tryptophan (Trp), cysteine (Cys), methionine (Met), serine (Ser), threonine (Thr), lysine (Lys), arginine (Arg), histidine (His), aspartatic acid (Asp), glutamic acid (Glu), asparagine (Asn), glutamine (Gln), proline (Pro), 4-hydroxy-proline (Hyp), 5-hydroxy-lysine, norleucine (Nle), 5-hydroxynorleucine (Hyn), 6-hydroxynorleucine, ornithine, or cyclohexylglycine (Chg) and wherein the N-terminal amino function of said aminoalkanoyl or oligopeptidoyl group is attached to a capping group Cg, most preferably wherein the unit A is derived from L-proline, glycine, L-norleucine, L-cyclohexylglycine, L-5-hydroxynorleucine, L-6-hydroxynorleucine, L-5-hydroxylysine, L-arginine, or L-lysine.

Most preferred are the compounds of formula I, wherein R¹ is a group selected from the formulae (1) to (14):

Cg-Gly (1)

Cg-Nle (2)

Cg-Val (3)

Cg-Met (4)

Cg-Xxx-Gly (5)

Cg-Xxx-Hyn (6)

Cg-Xxx-Pro (7)

Cg-Xxx-His (8)

Cg-Xxx-Met (9)

Cg-Xxx-Ala (10)

Cg-Xxx-Hyn (11)

Cg-Xxx-Ala-Gly (12)

Cg-(Xxx)ₙ—Xxx-Gly (13)

Cg-(Xxx)ₙ—Xxx-Ala-Gly (14)

wherein

Cg represents a capping group selected from pyridinyloxycarbonyl, pyridinylacetyl, pyridinylmethylsulfonyl and pyridylmethylaminocarbonyl;

Xxx represents a moiety derived from an amino carboxylic acid; and

n is an integer from 1 to 6.

In another preferred embodiment of the present invention the heterocyclic ring formed by R^{a}, R^{b} and the interjacent N—C is substituted by R⁸ and R⁹, wherein R⁸ and R⁹ each independently represent a hydrogen or halogen atom or a C₁-C₆-alkyl, C₁-C₆-alkylamino, di-C₁-C₆-alkylamino, C₁-C₆-alkoxy, thiol, C₁-C₆-alkylthio, oxo, imino, fomyl, C₁-C₆-alkoxy carbonyl, amino carbonyl, C₃-C₈-cycloalkyl, aryl, or heteroaryl group.

Most preferred are the compounds of fomula IA

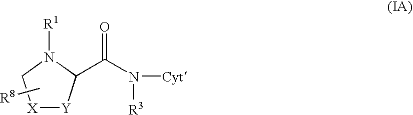

wherein R¹, R³, R⁸, Cyt′ are defined as hereinabove and hereinbelow, and X-Y represents CHR⁹—CH₂, CR²═CH, NH—CH₂, CH₂—NH, —CR⁹—, CH₂—CHR⁹—CH₂, in particular compounds of fomulae IA1 or IA2,

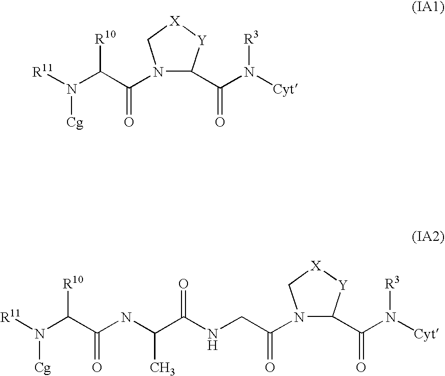

wherein R³, Cyt′ Cg, X and Y are as defined in any of the preceding claims, and R¹⁰ and R¹¹ each independently represent a hydrogen atom, an optionally substituted C₁-C₆-alkyl, C₃-C₈-cycloalkyl, aryl or heteroaryl group or

R¹⁰ and R¹¹ together with the interjacent N—C group form an optionally substituted, optionally benzo- or cyclohexano-condensed 3- to 7-membered saturated or unsaturated heterocyclic ring, in which one or two CH₂ groups may also be replaced by NH, O or S.

In another preferred embodiment of the present invention R² represents a group selected from

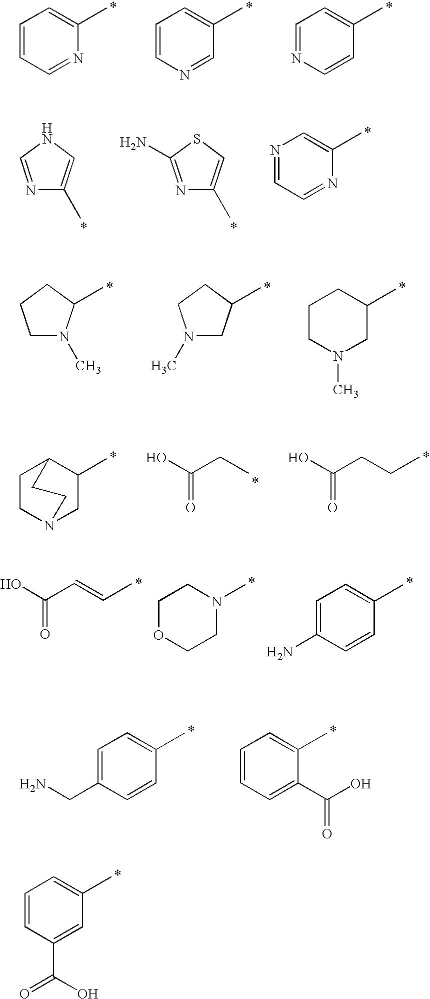

Unless indicated otherwise, the simple stereoisomers as well as mixtures or racemates of the stereoisomers are included in the invention.

“C₁-C₆-alkyl” generally represents a straight-chained or branched hydrocarbon radical having 1 to 6 carbon atoms.

The term “optionally substituted” as used hereinabove or hereinbelow with respect to a group or a moiety refers to a group or moiety which may optionally be substituted by one or several halogen atoms, hydroxyl, amino, C₁-C₆-alkyl-amino, di-C₁-C₆-alkyl-amino, C₁-C₆-alkyl-oxy, thiol, C₁-C₆-alkyl-thio, ═O, ═NH, —CHO, —COOH, —CONH₂, —NHC(═NH)NH₂, C₃-C₈-cycloalkyl, aryl, or heteroaryl substituents, which may be identical to one another or different.

The following radicals may be mentioned by way of example:

Methyl, ethyl, propyl, 1-methylethyl (isopropyl), butyl, 1-methylpropyl, 2-methylpropyl, 1,1-dimethylethyl, n-pentyl, 1-methylbutyl, 2-methylbutyl, 3-methylbutyl, 1,1-dimethyl-propyl, 1,2-dimethylpropyl, 2,2-dimethylpropyl, 1-ethylpropyl, hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2methyl-propyl, HOCH₂—, CH₃CH(OH)—, CH₃CH(OH)CH₂CH₂—, HOCH₂CH₂CH₂CH₂—, H₂NCH₂CH₂CH₂—, H₂NCH₂CH₂CH₂CH₂—, H₂NCH₂CH(OH)CH₂CH₂—, H₂NC(═NH)NHCH₂CH₂CH₂—, HSCH₂—, CH₃SCH₂CH₂—, HOOCCH₂—, HOOCCH₂CH₂—, H₂NC(═O)CH₂—, H₂NC(═O)CH₂CH₂—, benzyl, para-hydroxy-benzyl,

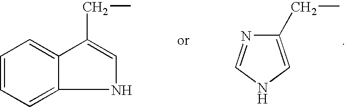

If a C₁-C₆-alkyl group is substituted, the substituents are preferably hydroxyl, amino, dimethylamino, diethylamino, thiol, methyl-thiol, methoxy, ethoxy, ═O, ═NH, —CHO, —COOH, —COOCH₃, —COOCH₂CH₃, —CONH₂, —NHC(═NH)NH₂, cyclohexyl, phenyl, benzyl, para-hydroxy-benzyl,

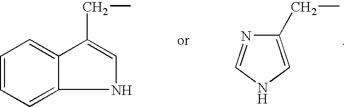

If C₁-C₆-alkyl is substituted with aryl or heteroaryl, C₁-C₆-alkyl is preferably C₁, more preferably a methylene group.

The terms “amino alkanoyl” and “oligopeptidoyl” including “di- or tripeptidoyl” as used hereinabove or hereinbelow with respect to radical R¹ describe a radical in which an amino acid or an oligomer comprising up to 12, preferably 2 or 3 amino acid moieties is attached C-terminally to the nitrogen atom of the heterocyclic ring via an amide bond.

A person of ordinary skill in the chemistry of amino acids and oligopeptides will readily appreciate that certain amino acids may be replaced by other homologous, isosteric and/or isolectronic amino acids wherein the biological activity of the original amino acid or oligopeptide has been conserved upon modification. Certain unnatural and modified natural amino acids may also be utilized to replace the corresponding natural amino acid. Thus, for example, tyrosine may be replaced by 3-iodotyrosine, 2- or 3-methyltyrosine, 3-fluorotyrosine.

The term “capping group” as used hereinabove or hereinbelow with respect to a group which is attached to the N-terminal nitrogen atom of the amino alkanoyl or oligopeptidoyl group of radical R¹ defines a group or moiety which reduces or eliminates the enzymatic degradation of the compounds of the present invention by the action of amino peptidases which are present in the blood plasma of warm blooded animals and enhances the physical stability of an aqueous pharmaceutical formulations comprising said prodrug.

“C₃-C₈-Cycloalkyl” generally represents cyclic hydrocarbon radical having 3 to 8 carbon atoms which may optionally be substituted by one or several hydroxyl, amino, C₁-C₆-alkyl-amino, di-C₁-C₆-alkyl-amino, C₁-C₆-alkyl, C₁-C₆-alkyloxy, thiol, C₁-C₆-alkyl-thio, ═O, ═NH, —CHO, —COOH, —COOCH₃, —COOCH₂CH₃, —CONH₂, —NHC(═NH)NH₂, or halogen substituents, which may be identical to one another or different.

“Heterocyclic ring” as used hereinabove and hereinbelow with respect to the group formed by R^{a} and R^{b} together with the interjacent N—C group generally represents a 3 to 7-membered, preferably 4-, 5- or 6-membered non-aromatic heterocyclic ring system, containing one nitrogen atom and optionally 1 or 2 additional heteroatoms selected from the group of nitrogen, oxygen and sulfur, which may be substituted by one or several halogen atoms or C₁-C₆-alkyl, C₁-C₆-alkylamino, di-C₁-C₆-alkylamino, C₁-C₆-alkoxy, thiol, C₁-C₆-alkylthio, oxo, imino, fomyl, C₁-C₆-alkoxy carbonyl, amino carbonyl, C₃-C₈-cycloalkyl, aryl, or heteroaryl groups, which may be identical to one another or different, and which optionally may be benzo- or cyclohexano-condensed. Such heterocyclic rings are preferably azetidine or are derived from a fully or partially hydrogenated pyrrole, pyridine, thiazole, isoxazole, pyrazole, imidazole, indole, benzimidazole, indazole, pyridazine, pyrimidine, pyrazin group. Most preferred are azetidine, pyrrolidine, 3,4-dehydropyrrolidine, piperidine, hexahydro-1H-azepine, octahydroindole, imidazolidine, thiazolidine.

If such heterocyclic ring is substituted, the substituents are preferably methyl, ethyl, propyl, 1-methylethyl (isopropyl), butyl, 1-methylpropyl, 2-methylpropyl, 1,1-dimethylethyl, hydroxyl, amino, dimethyl-amino, diethyl-amino, thiol, methyl-thiol, methoxy, ethoxy, —CHO, —COOH, —COOCH₃, —COOCH₂CH₃, or —CONH₂.

“Aryl” generally represents an aromatic ring system with 6 to 10, preferably 6 carbon atoms which may optionally be substituted by one or several hydroxyl, amino, C₁-C₆-alkyl-amino, di-C₁-C₆-alkyl-amino, C₁-C₆-alkyl, C₁-C₆-alkyloxy, thiol, C₁-C₆-alkyl-thio, —CHO, —COOH, —COOCH₃, —COOCH₂CH₃, —CONH₂, or halogen substituents, which may be identical to one another or different, and which optionally may be benzocondensed. Aryl substituents may be preferably derived form benzene, preferred examples being phenyl, 2-hydroxy-phenyl, 3-hydroxy-phenyl, 4-hydroxy-phenyl, 4-amino-phenyl, 2-amino-phenyl, 3-amino-phenyl.

If aryl is substituted, the substituents are preferably methyl, ethyl, propyl, 1-methylethyl (isopropyl), butyl, 1-methylpropyl, 2-methylpropyl, 1,1-dimethylethyl, hydroxyl, amino, dimethyl-amino, diethyl-amino, thiol, methyl-thiol, methoxy, ethoxy, —CHO, —COOH, —COOCH₃, —COOCH₂CH₃, or —CONH₂.

“Heteroaryl” generally represents a 5 to 10-membered aromatic heterocyclic ring system, containing 1 to 5 heteroatoms selected from the group of nitrogen, oxygen, or sulfur, which may optionally be substituted by one or several hydroxyl, amino, C₁-C₆-alkyl-amino, di-C₁-C₆-alkyl-amino, C₁-C₆-alkyl, C₁-C₆-alkyloxy, thiol, C₁-C₆-alkyl-thio, —CHO, —COOH, —COOCH₃, —COOCH₂CH₃, —CONH₂, or halogen substituents, which may be identical to one another or different, and which optionally may be benzocondensed. Heteroaryl substituents may preferably be derived from furane, pyrrole, thiophene, pyridine, thiazole, isoxazole, pyrazole, imidazole, benzofuran, thianaphthene, indole, benzimidazole, indazole, quinoline, pyridazine, pyrimidine, pyrazine, chinazoline, pyrane, purine, adenine, guanine, thymine, cytosine, uracil.

If heteroaryl is substituted, the substituents are preferably methyl, ethyl, propyl, 1-methylethyl (isopropyl), butyl, 1-methylpropyl, 2-methylpropyl, 1,1-dimethylethyl, hydroxyl, amino, dimethyl-amino, diethyl-amino, thiol, methyl-thiol, methoxy, ethoxy, —CHO, —COOH, —COOCH₃, —COOCH₂CH₃, or —CONH₂.

“Residue of a cytotoxic or cytostatic compound” means that the compound H₂N-Cyt′, which is released upon cleavage of the amide bond shown in formula (I), is either cytotoxic or cytostatic itself, or may be converted into a cytotoxic or cytostatic compound in a subsequent step.

In the latter case, -Cyt′ may be a residue of formula -L-Cyt″, wherein L is a linker residue derived from a bifunctional molecule, for instance a diamine H₂N-L′-NH₂, an amino alcohol H₂N-L′-OH, for example p-amino-benzyl alcohol (PABOH), an amino carbonate, for example

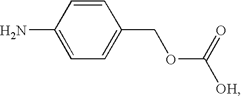

or an unnatural amino carboxylic acid. If -Cyt′ is of formula -L-Cyt″, the compound H₂N-L′-Cyt″ is generated by the enzymatic cleavage of the amide bond shown in formula (I). The compound H₂N-L′-Cyt″ may be cytotoxic or cytostatic itself or the linker residue cleaved off from Cyt″ in a subsequent step releasing the cytotoxic or cytostatic agent. For example, the compound H₂N-L′-Cyt″ may be hydrolysed under physiological conditions into a compound H₂N-L′-OH and the cytotoxic or cytostatic compound H-Cyt″, which is the active therapeutic agent (In the following, only the term Cyt′ is used for both Cyt′ and Cyt″, and only the term L is used for both L and L′, for simplicity).

The pharmaceutically acceptable salts of the compounds of the present invention include the conventional non-toxic salts formed from non-toxic inorganic or organic acids. For example, such conventional non-toxic salts include those from inorganic acids such as hydrochloric acid, hydrobromic, sulfuric, sulfamic, phosphoric, nitric and the like; and the salts prepared from organic acids such as acetic, propionic, succinic, glycolic, stearic, lactic, malic, tartaric, citric, ascorbic, maleic, phenylacetic, glutamic, benzoic, salicylic, sulfanilic, oxalictrifluoroacetic and the like.

H₂N-Cyt′ is preferably an anthracycline derivative of formula II

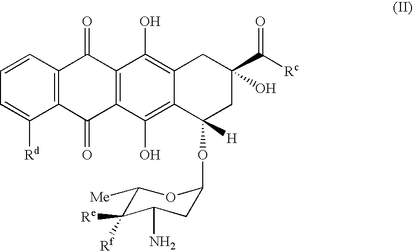

wherein

R^{c} represents C₁-C₆ alkyl, C₁-C₆ hydroxyalkyl or C₁-C₆ alkanoyloxy C₁-C₆ alkyl, in particular methyl, hydroxymethyl, diethoxyacetoxymethyl or butyryloxymethyl;

R^{d} represents hydrogen, hydroxy or C₁-C₆ alkoxy, in particular methoxy;

one of R^{e} and R^{f} represents a hydrogen atom; and the other represents a hydrogen atom or a hydroxy or tetrahydropyran-2-yloxy (OTHP) group.

Paricularly preferred are the following compounds of formula II:

| R^{c} | R^{d} | R^{e} | R^{f} | Cyt |
|---|---|---|---|---|
| CH₂OH | OCH₃ | H | OH | doxorubicin |
| CH₃ | OCH₃ | H | OH | daunorubicin |
| CH₂OH | OCH₃ | OH | H | epirubicin |
| CH₃ | H | H | OH | idarubicin |
| CH₂OH | OCH₃ | H | OTHP | THP |
| CH₂OH | OCH₃ | H | H | esorubicin |
| CH₂OCOCH(OC₂H₅)₂ | OCH₃ | H | OH | detorubicin |
| CH₂OH | H | H | OH | carminorubicin |
| CH₂OCOC₄H₉ | OCH₃ | H | OH | |

Most preferred is doxorubicin (Dox). Other cytotoxic or cytostatic residues Cyt′ may be derived for example from methotrexate, trimetrexate, pyritrexim, 5,10-dideazatetrahydrofolatepyrimetamine, trimethoprim, 10-propargyl-5,8-dideazafolate2,4-diamino-5(3′,4′-dichloropheyl)-6-methylpyrimidine, aminoglutethimide, goreserelin, melphalan, chlorambucil, analogs of other chemotherapeutic agents such as 9-aminocamtothecin (for examples see e.g. Burris H A, r. d. and S. M. Fields (1994). “Topoisomerase I inhibitors. An overview of the camptothecin analogs. [Review].” Hematol. Oncol. Clin. North Am. 8(2): 333-355; Iyer, L. and M. J. Ratain (1998). “Clinical pharmacology of camptothecins. [Review] [137 refs].” Cancer Chemother. Pharmacol. 42 Suppl: S31-S43.)

In formula (I), Cyt′ may also be a biological effector molecule which either directly or indirectly effects destruction of tumor cells, like for example TNFα.

Preferred anthracycline prodrugs are the compounds of formula III

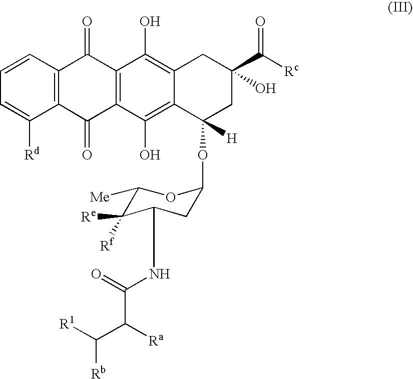

wherein R^{a}, R^{b}, R^{c}, R^{d}, R^{e}, R^{f} and R¹ are as defined hereinabove.

Most preferred compounds of the invention are doxorubicin derivatives of formulae (IIIA) to (IIIH):

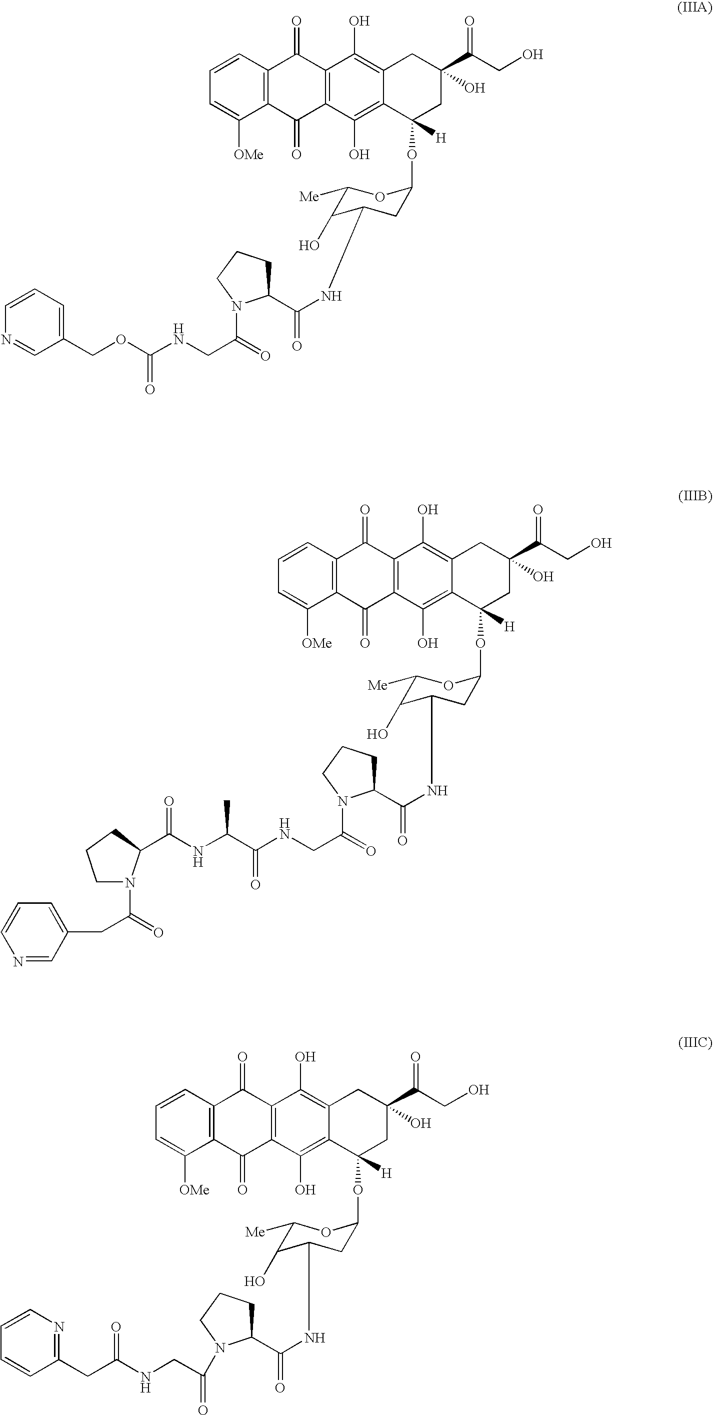

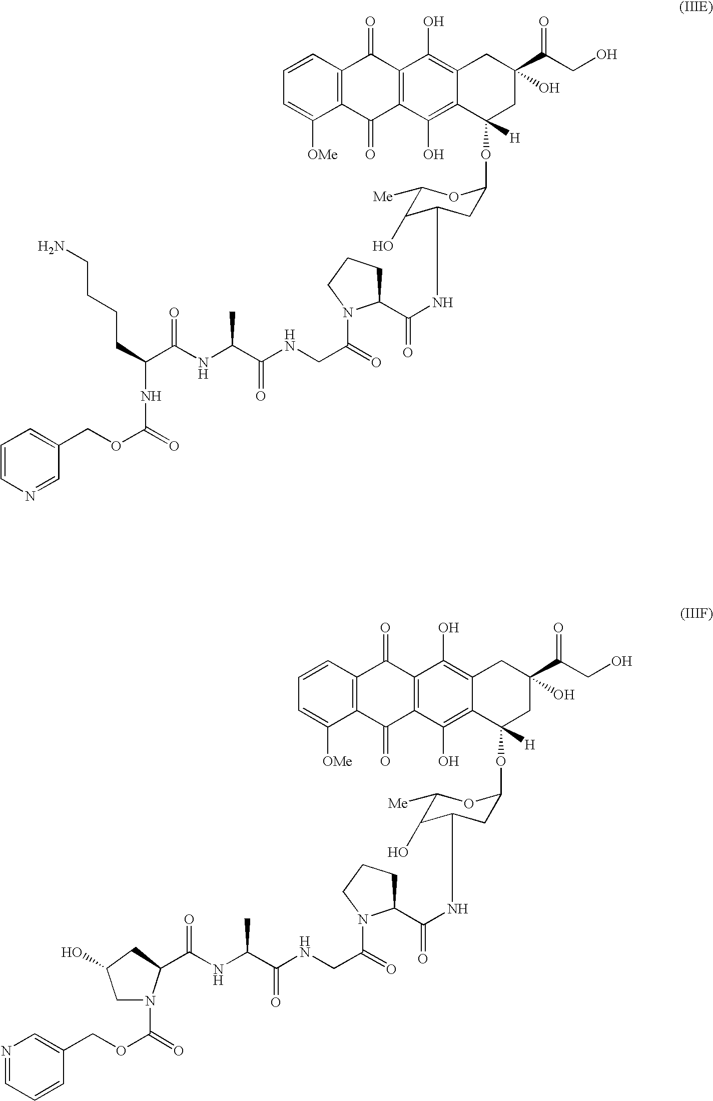

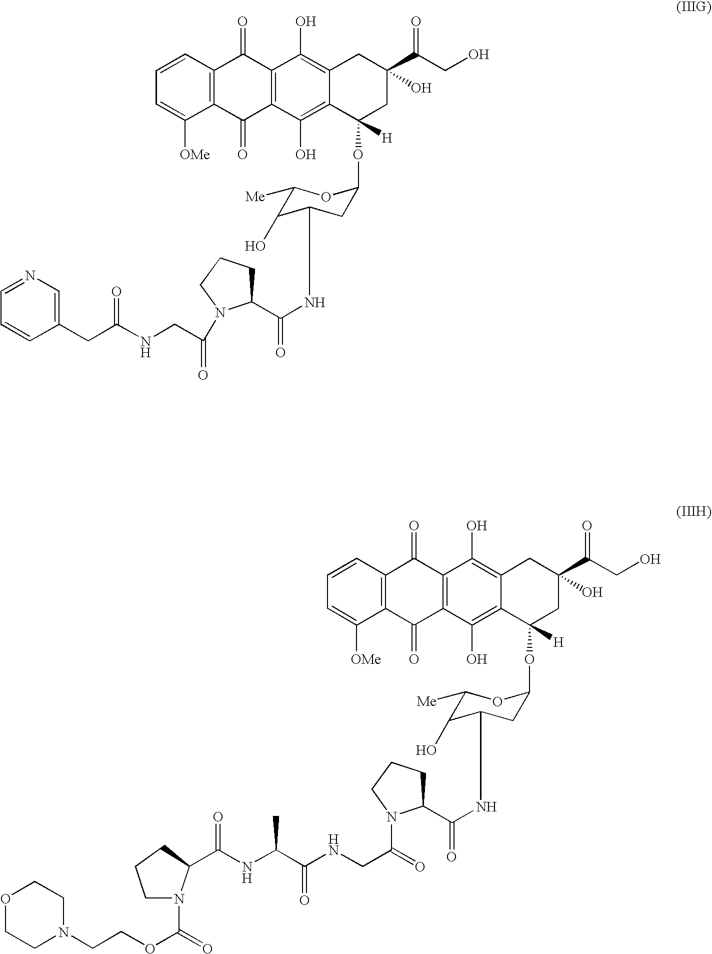

If the part Cg-B-A or Cg-(D)ₘ-B-A of formula (I) contains two or more sulfur atoms, the compound of the invention may contain one or more disulfide bonds.

One class of cytotoxic or cytostatic compounds which may be used for the present invention has a primary amino function which is available for formation of an amidic bond as shown in formula (I), like doxorubicin. In this case, a linker molecule L is not necessary. If a cytostatic or cytotoxic compound does not have such an amino function, such a function may be created in such a compound by way of chemical modification, e.g. by introducing or converting a functional group or attaching a linker molecule to the compound. A linker molecule may also be inserted between the oligomeric part (i.e. the part comprising the amino carboxylic residues) and the cytostatic or cytotoxic part of the compound of the invention to ensure or optimise cleavage of the amide bond between the oligomeric part and the cytotoxic or cytostatic part. If a linker molecule is present, i.e. in compounds containing the structure L-Cyt′, the bond between L and Cyt′ is preferably an amidic or ester bond. In a preferred embodiment, such a linker molecule is hydrolysed off the cytostatic or cytotoxic compound under physiological conditions after the enzymatic cleavage and thus the free cytostatic or cytotoxic compound is generated. In any case, the compound of the invention must have the property of being cleavable upon the catalytic action of FAPα and, as a direct or indirect consequence of this cleavage, releasing under physiological conditions a cytostatic or cytotoxic compound.

In a further aspect, the present invention relates to prodrug that is capable of being converted into a cytotoxic or cytostatic drug, by the catalytic action of FAPα, said prodrug exhibits an oligomeric part comprising up to 13 amino carboxylic residues, the C-terminal amino carboxylic thereof is recognised by FAPα, and a cytotoxic or cytostatic part, characterized in that the N-terminal amino function of the oligomeric part is attached to a capping group (Cg) which is capable of enhancing the chemical stability of said prodrug under physiological conditions and the physical stability of an aqueous pharmaceutical formulations comprising said prodrug.

The oligomeric part is preferably a peptide. Preferably, the oligomeric part comprises two, three, four, five, six, seven, eight, nine, ten, eleven, or twelve amino carboxylic acid residues, more preferably two, three, or four amino carboxylic residues. The N-terminal amino function is preferably protected by a capping group.

The compounds of the invention may be synthesized by processes known in the art (E. Wünsch, Synthese von Peptiden, in “Methoden der organischen Chemie”, Houben-Weyl (Eds. E. Müller, O. Bayer), Vol. XV, Part 1 and 2, Georg Thieme Verlag, Stuttgart, 1974). For example, the compounds could be synthesized in a block synthetic fashion by condensation of the terminal carboxy function of the oligomeric part, wherein X may be OH or an activation leaving group, with the amino group of the cytotoxic or cytostatic molecule H₂N-Cyt′ resulting in an amide formation.

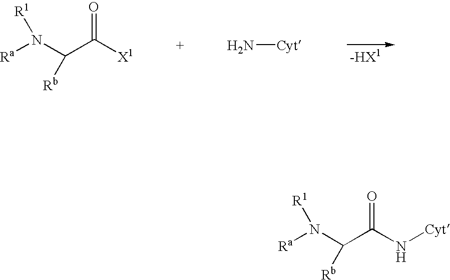

If a linker residue (L) is required between the oligomeric part and the cytotoxic or cytostatic agent the block synthesis can be done in the same manner.

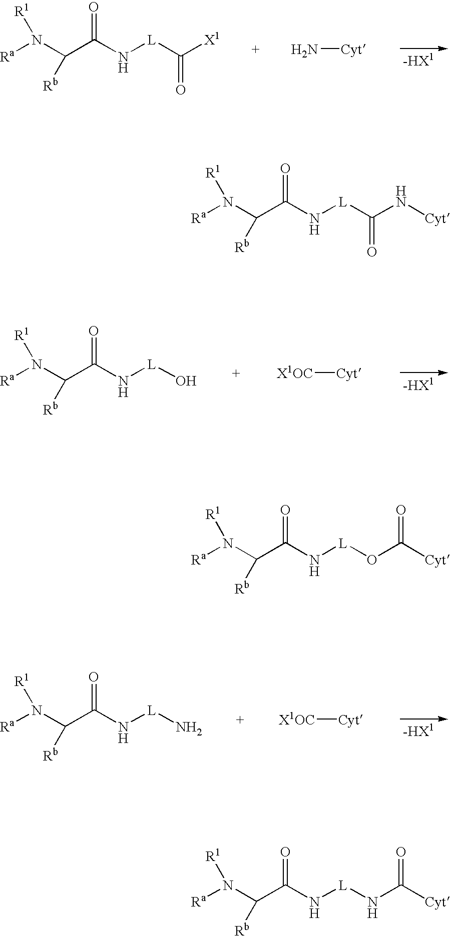

If the cytotoxic or cytostatic bears a carboxy function for the attachment to the oligomeric part, the linker molecule can be an amine or an amino alcohol and the block synthesis of such compounds can be carried out in a similar way by reaction of the activated XOC-Cyt′ with either the hydroxy or the amino component.

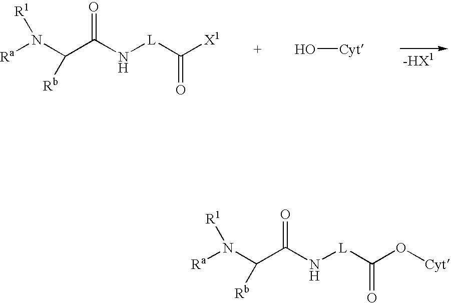

If the cytotoxic or cytostatic reagent has a hydroxy function which is suitable for the coupling to the oligomeric part the linker residue may be an amino carboxylic acid and a block synthesis can be done similarly.

If necessary, other functional groups in the units Cyt′, L, hydroxyproline, A, B and D which shall not react during the assembly of the target molecules may be protected by suitable protecting groups. Suitable protecting groups are well known from the state of the art (P. G. M. Wuts, “Protective groups in organic synthesis”, John Wiley and Sons Inc., New York 1991). These protecting groups are removed at the end of the synthesis.

By way of example only, useful amino-protecting groups may include, for example, C₁-C₁₀ alkanoyl groups such as formyl, acetyl dichloroacetyl, propionyl, 3,3-diethylhexanoyl, and the like, C₁-C₁₀alkoxycarbonyl and C₆-C₁₇ aralkyloxycarbonyl groups such as tert-butoxycarbonyl (BOC), benzyloxycarbonyl, fluorenylmethoxycarbonyl, and the like. Most preferred is fluorenylmethoxycarbonyl (FMOC).

Suitable carboxy-protecting groups may include, for example, C₁-C₁₀ alkyl groups such as methyl, tert-butyl, decyl; C₆-C₁₇ aralkyl such as benzyl, 4-methoxybenzyl, diphenylmethyl, triphenylmethyl, fluorenyl; tri-(C₁-C₁₀ alkyl)silyl or (C₁-C₁₀ alkyl)diarylsilyl such as trimethylsilyl, dimethyl-tert-butylsilyl, diphenyl-tert-butylsilyl and related groups.

To achieve such ester- or amide formations, it may be necessary to activate the carbonyl group of the carboxylic acid for a nucleophilic attack of an amine or alcohol, i.e. X to be an activation group or leaving group which is suitable to be substituted by an amino group. This activation can be done by conversion of the carboxylic acid into an acid chloride or acid fluoride or by conversion of the carboxylic acid into an activated ester, for instance a N-hydroxysuccinimidyl ester or a pentafluorophenyl ester. Another method of activation is the transformation into a symmetrical or unsymmetrical anhydride. Alternatively, the formation of the amide- or ester bonds can be achieved by the use of in situ coupling reagents like benzotriazole-1-yl-oxy-tris-pyrrolidino-phosphonium hexafluorophosphate (PyBOP) (E. Frerot et al., Tetrahedron, 1991, 47, 259-70), 1,1′-carbonyldimidazole (CDI) (K. Akaji et al., THL, 35, 1994, 3315-18), 2-(1H-benzotriazole-1-yl)-1,1,3,3-tetramethyluronium tetrafluoroborate (TBTU) (R. Knorr et al., THL, 30, 1989, 1927-30), 1-(mesitylene-2-sulfonyl)-3-nitro-1H-1,2,4-triazole (MSNT) (B. Blankenmeyer-Menge et al., THL, 31, 1990, 1701-04).

As an alternative to the block synthesis the molecules in the general formula (I) can be assembled in a step by step manner starting at the right hand side by stepwise condensation reactions of the respective monomers Cyt′, L, the cyclic amino acid group formed by R^{a}, R^{b} and the interjacent N—C group, in particular proline or hydroxyproline, A, B and D. For the condensation reaction the same above mentioned coupling methods can be applied. Since the units L, proline/hydroxyproline, A, B and D are at least bifunctional molecules containing an amino- and (at least the units A, B, D, and the cyclic amino acid group formed by Ra, Rb and the interjacent N—C group, in particular proline/hydroxyproline) a carboxy group, the amino group needs to be blocked by a protecting group (PG) prior to the activation of the carboxylic function. For the protection of the amino groups the group BOC or preferably the group FMOC can be applied. After the coupling reaction the amino protecting group has to be removed and the coupling with the next Fmoc- or Boc-protected unit can be carried out. If necessary, other functional groups in the units Cyt′, L, the cyclic amino acid group formed by Ra, Rb and the interjacent N—C group, in particular hydroxyproline, A, B and D which shall not react during the assembly of the target molecules may be protected by suitable protecting groups. These protecting groups are removed at the end of the synthesis.

Capping groups as defined in the context of formula (I) may also serve as protection groups, in particular when the last (N-terminal) amino carboxylic acid unit is added. In this latter case the protecting group is not removed as it is a part of the target molecule. Alternatively, the capping group may be added after the last amino carboxylic acid unit has been coupled and deprotected.

The step by step synthesis is outlined in the following schemes. The second scheme is exemplary as the linker residue as well as the Cyt′ residue may contain other functional groups as indicated in this scheme (see above):

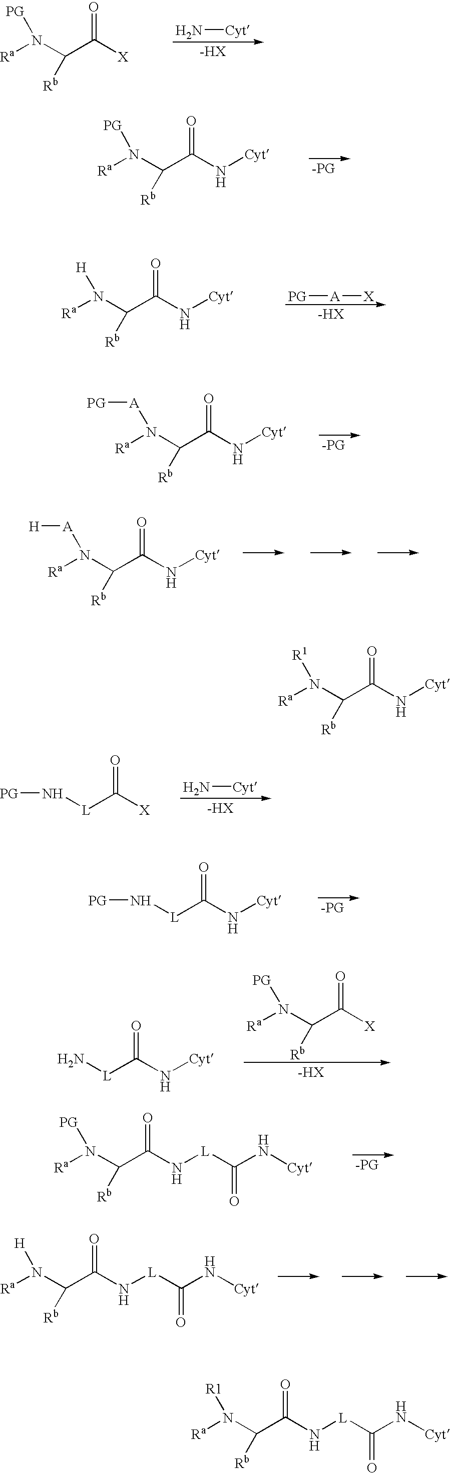

Preferably, X is a leaving group, for example —Cl, —F, N-hydroxysuccinimidyl, pentafluorophenyl, or a carboxylate. Alternatively, X² may be OH and condensation is achieved by the use of an in situ coupling reagent, for example benzotriazole-1-yl-oxy-tris-pyrrolidino-phosphonium hexafluorophosphate (PyBOP), 1,1′-carbonyldimidazole (CDI), 2-(1H-benzotriazole-1-yl)-1,1,3,3-tetramethyluronium tetrafluoroborate (TBTU), or 1-(mesitylene-2-sulfonyl)-3-nitro-1H-1,2,4-triazole (MSNT).

PG is a protecting group for example BOC, or preferably FMOC.

The compounds of the invention are intended for medical use. In particular, these compounds are useful for the treatment of tumors which are associated with stromal fibroblasts that express FAPα and which are generally not optimally treated with available cytotoxic and/or cytostatic agents. Tumors with this property are, for example, epithelial cancers, such as lung, breast, and colon carcinomas. Tumors, such as bone and soft tissue sarcomas which express FAPα, may also be treated with these compounds.

Consequently, another aspect of the present invention are pharmaceutical compositions comprising a compound of the present invention and optionally one or more suitable and pharmaceutically acceptable excipients, as exemplified in: Remington: the science and practice of pharmacy. 19th ed. Easton: Mack Publ., 1995. The pharmaceutical compositions may be formulated as solids or solutions. Solid formulations may be for preparation of a solution before injection. Preferably, the pharmaceutical compositions of the invention are solutions for injection. They may be administered systemically, e.g. by intravenous injection, or topically, e.g. by direct injection into the tumor site. The dosage will be adjusted according to factors like body weight and health status of the patient, nature of the underlying disease, therapeutic window of the compound to be applied, solubility, and the like. It is within the knowledge of the expert to adjust dosage appropriately. For doxorubicin conjugates, for example, the dose will preferably be in the range from 10 mg/m² to 2000 mg/m², but also higher or lower doses may be appropriate.

Accordingly, a further aspect of the present invention is the use of a compound of the invention in the preparation of a pharmaceutical composition for the treatment of cancer. Furthermore, an aspect of the invention is a method of treatment of cancer, comprising administering an effective amount of a pharmaceutical composition of the invention to a patient. Indications include the treatment of cancer, specifically:

1) The treatment of epithelial carcinomas including breast, lung, colorectal, head and neck, pancreatic, ovarian, bladder, gastric, skin, endometrial, ovarian, testicular, esophageal, prostatic and renal origin;

2) Bone and soft-tissue sarcomas: Osteosarcoma, chondrosarcoma, fibrosarcoma, malignant fibrous histiocytoma (MFH), leiomyosarcoma;

3) Hematopoietic malignancies: Hodgkin's and non-Hodgkin's lymphomas;

4) Neuroectodermal tumors: Peripheral nerve tumors, astrocytomas, melanomas;

5) Mesotheliomas.

Also included are the treatment of chronic inflammatory conditions such as rheumatoid arthritis, osteoarthritis, liver cirrhosis, lung fibrosis, arteriosclerosis, and abnormal wound healing.

A further aspect of the invention is a method of treatment of cancer, wherein a prodrug is administered to a patient wherein said prodrug is capable of being converted into a cytotoxic or cytostatic drug by an enzymatic activity, said enzymatic activity being the expression product of cells associated with tumor tissue. Preferably, said enzymatic activity is the proteolytic activity of FAPα.

One method of administration of the compounds is intravenous infusion. Other possible routes of administration include intraperitoneal (either as a bolus or infusion), intramuscular or intratumoral injection. Where appropriate, direct application may also be possible (for example, lung fibrosis).

One skilled in the art will appreciate that although specific reagents and reaction conditions are outlined in the following examples, modifications can be made which are meant to be encompassed by the scope of the invention. The following examples, therefore, are intended to further illustrate the invention and are not limiting.

### EXAMPLE 1

Synthesis of 3,5-Difluorophenylacetyl-Gly-Pro-OH

H-Pro-2-chlorotritylchloride-resin (200 mg, 0.158) was added to a reaction vessel and washed with DMF (three times with 7 ml). Fmoc-Gly-OH (284.4 mg, 0.95 mmol), HOBt (128.1, 0.95 mmol), DIC (146.8 μl, 0.95 mmol) and DMF (6 ml) were added to the reaction vessel. After 6 h of agitation, the resin was then filtered, washed with DMF (eight times with 7 ml) and 20% piperidine in DMF (4 ml) was added to the reaction vessel. After agitation for 30 min, the resin was filtered and washed with DMF (eight times with 7 ml). 3,5-Difluorophenylacetic acid (163.2 mg, 0.95 mmol), 1-hydroxybenzotriazole (HOBt) (128.1, 0.95 mmol), diisopropylcarbodiimide (DIC) (146.8 μl, 0.95 mmol) and N,N dimethylformamide (DMF) (6 ml) were added to the reaction vessel. After 12 h of agitation, the resin was washed with DMF (six times with 7 ml), dichloromethane (DCM) (six times with 7 ml), methanol (MeOH) (six times with 7 ml), and Et₂O (six times with 7 ml) and treated with a solution of trifluoroacetic acid/water 95:5 (6 ml). After incubation for 2 h, the cleavage solution was placed into a flask and the resin was washed additionally with DCM (twice with 3 ml).

The cleavage solution was removed with a roto-evaporator and the resulting oil was dried with a stream of nitrogen. The crude product was purified by preparative reversed phase HPLC applying a acetonitrile/water gradient. The product gave satisfactory analytical data. HPLC>95%; ES-MS: m/z=326.3 ([M+H]⁺)

### EXAMPLES 2 TO 6

The following N-terminal blocked peptides were prepared analogously to this method:

| Example | Compound |
|---|---|
| 2 | 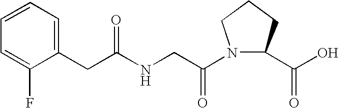 |
| 3 | 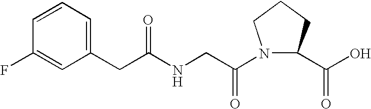 |
| 4 | 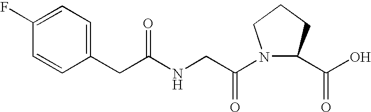 |
| 5 | 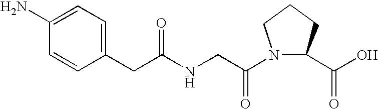 |
| 6 | 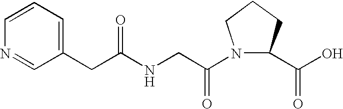 |

### EXAMPLE 7

Synthesis of Pyridin-3-ylmethoxycarbonyl-Pro-Ala-Gly-Pro-OH

H-Pro-2-chlorotritylchloride-resin (200 mg, 0.158) was added to a reaction vessel and washed with DMF (three times with 7 ml). Fmoc-Gly-OH (284.4 mg, 0.95 mmol), HOBt (128.1, 0.95 mmol), DIC (146.8 μl, 0.95 mmol) and DMF (6 ml) were added to the reaction vessel. After 6 h of agitation, the resin was then filtered, washed with DMF (eight times with 7 ml) and 20% piperidine in DMF (4 ml) was added to the reaction vessel. After agitation for 30 min, the resin was filtered and washed with DMF (eight times with 7 ml). Fmoc-Ala-OH and Fmoc-Pro-OH were incorporated in the same manner. The Fmoc-group of proline was removed and the resin was washed with DMF (eight times with 7 ml) and DCM (six times with 7 ml). 3-Pyridylcarbinol (99 μl, 0.25 mmol) was treated with 4-nitrophenyl chloroformate (156 mg, 0.20 mmol) and triethylamine (225 μl, 0.4 mmol) in DCM (7 ml) for 6 h and the resulting mixture was added to the resin. After 12 h of agitation, the resin was washed with DCM (six times with 7 ml), MeOH) (six times with 7 ml), and Et₂O (six times with 7 ml) and treated with a solution of trifluoroacetic acid/water 95:5 (6 ml). After incubation for 2 h, the cleavage solution was placed into a flask and the resin was washed additionally with DCM (twice with 3 ml).

The cleavage solution was removed with a roto-evaporator and the resulting oil was dried with a stream of nitrogen. The crude product was purified by preparative reversed phase HPLC applying a acetonitrile/water gradient. The product gave satisfactory analytical data. HPLC >95%; ES-MS: m/z=475.5 ([M+H]⁺)

### EXAMPLES 8 TO 20

The following blocked peptides have been prepared analogously.

| Example | Compound |
|---|---|
| 8 | 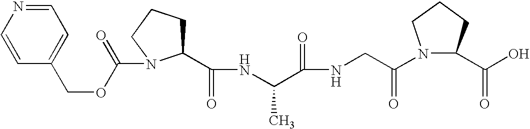 |
| 9 | 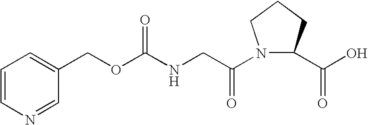 |
| 10 | 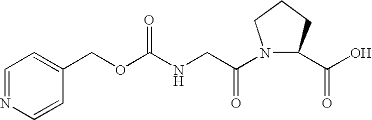 |
| 11 | 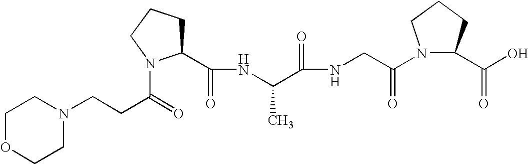 |
| 12 | 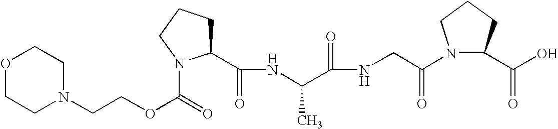 |
| 13 | 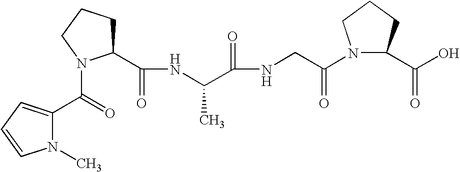 |
| 14 | 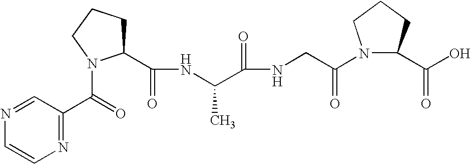 |
| 15 | 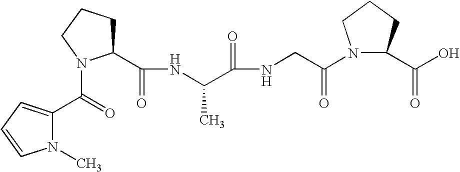 |
| 16 | 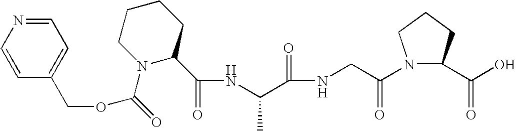 |
| 17 | 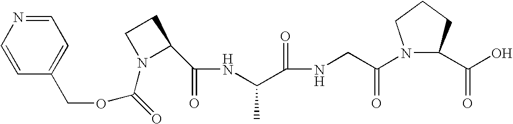 |
| 18 | 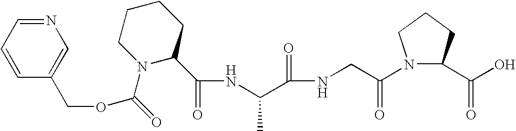 |
| 19 | 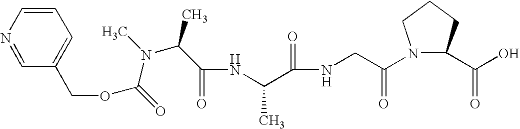 |
| 20 | 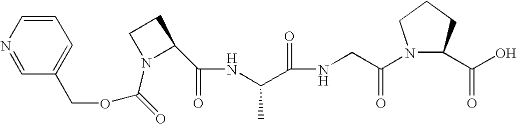 |

### EXAMPLE 21

Synthetic Procedures of Doxorubicin Conjugates

21 Pyridin-3-ylmethoxycarbonyl-Pro-Ala-Gly-Pro-Doxorubicin Pyridin-3-ylmethoxycarbonyl-Pro-Ala-Gly-Pro-OH (180.7 mg, 0.38 mmol) and N-hydroxysuccinimide (44 mg, 0.37 mmol) were weighed out and placed in a 2 neck-round bottom flask under dinitrogen. Anhydrous N,N-dimethylformamide (20 ml) was added and the flask was cooled to 0° C. in an ice bath. Dicyclohexylcarbodiimide (78 mg, 0.38 mmol) was added as a 1 ml solution in N,N-dimethylformamide. The solution was stirred at 0° C. for 40 minutes.

Doxorubicin.HCl (100 mg, 0.38 mmol) was weighed into a separate vial. N,N-dimethylformamide (3 ml) and NN-Diisopropylethylamine (33.1 μl, 0.19 mmol) were added to the vial with stirring. The doxorubicin solution was added via syringe to the peptide solution, and the vial was rinsed with an additional 2 ml of N,N-dimethylformamide. The ice bath was removed and reaction mixture was stirred for approximately 48 hours at room temperature.

The solvent was removed with a roto-evaporator and the resulting oil was dried with a stream of nitrogen. The crude product was purified by preparative reversed phase HPLC applying a acetonitrile/water gradient. The product gave satisfactory analytical data. HPLC>95%; ES-MS: m/z 1002 ([M+H]⁺)

### EXAMPLES 22 TO 40

Analogously are obtained the following doxorubicin conjugates of formula

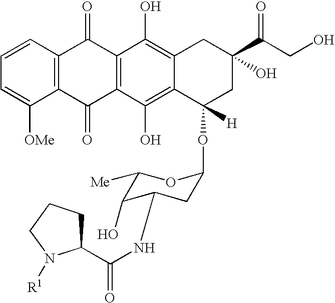

| Example | R¹ |
|---|---|
| 22 | 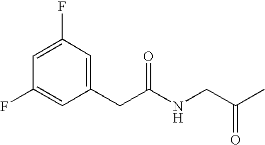 |
| 23 | 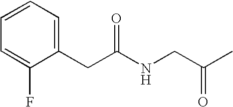 |
| 24 | 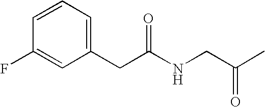 |
| 25 | 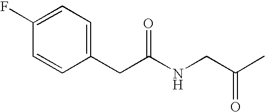 |
| 26 | 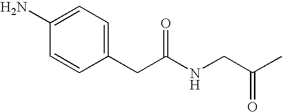 |
| 27 | 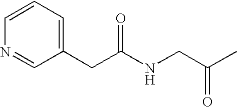 |
| 28 | 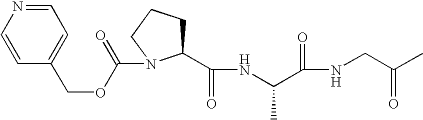 |
| 29 | 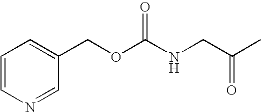 |
| 30 | 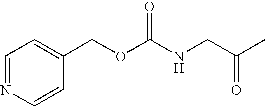 |
| 31 | 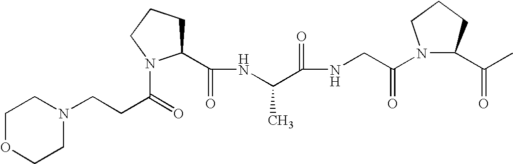 |
| 32 | 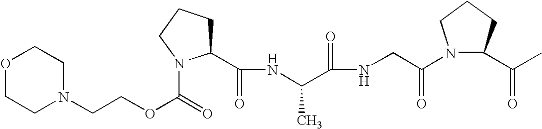 |
| 33 | 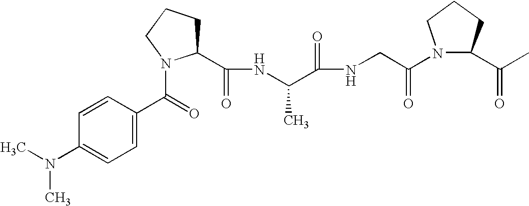 |
| 34 | 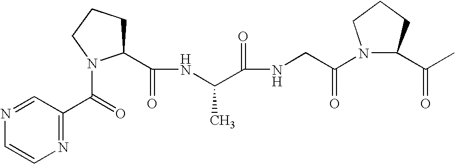 |
| 35 | 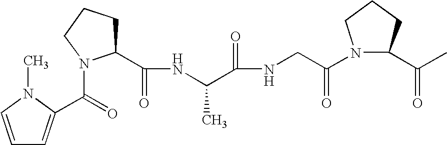 |
| 36 | 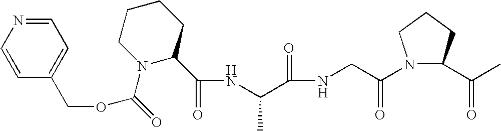 |
| 37 | 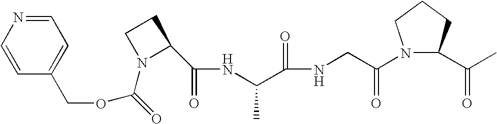 |
| 38 | 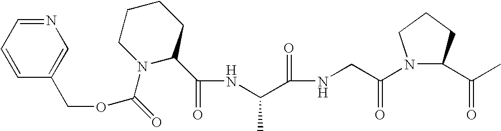 |
| 39 | 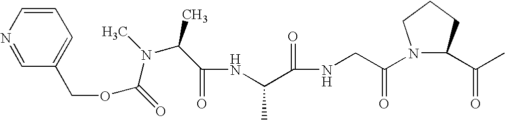 |
| 40 | 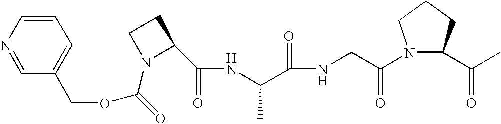 |

### EXAMPLE 41

Preparation of FAPα-Expressing Cell Lines

Mammalian cell lines expressing recombinant FAPα were prepared. HT1080 fibrosarcoma cells, widely known and available from the DSMZ (German Collection of Microorganisms and Cell Cultures, Braunschweig, Germany) under the accession number DSMZ ACC 315, were maintained in a DMEM/F12 mix 50:50 containing 10% fetal bovine serum in an atmosphere of 95% air and 5% CO₂. HT1080 cells were transfected with FAP.38 vector (WO 97/34927, Scanlan et al., loc. cit.) using the Lipofectin method according to the manufacturer's instructions (Gibco/BRL). Transfectants were selected for resistance to antibiotics (200 ug/ml Geneticin) and thereafter maintained in medium containing Geneticin. Individual colonies of resistant cells were picked, grown to confluence in 10 cm tissue culture petri dishes and tested for FAPα expression in an immunofluorescence assay using the FAPα-specific monoclonal antibody F19, as described (Garin-Chesa et al. (1990) Proc. Natl. Acad. Sci. USA 87(18), 7235-7239). The parental HT1080 cell line showed no detectable FAPα expression in this immunofluorescence assay, while one clone, referred to hereafter as HT1080 clone 33, was positive for FAPα.

Similarly, human embryonic kidney 293 cells, widely known and available from American Tissue Type Collection (Rockville, Md.), were maintained in a DMEM containing 10% fetal bovine serum in an atmosphere of 95% air and 5% CO₂. Cells were transfected with a FAPα expression vector, pFAP.38 using calcium phosphate transfection as described (Park, J. E., Chen, H. H., Winer, J., Houck, K. A. & Ferrara, N. (1994). Placenta growth factor. Potentiation of vascular endothelial growth factor bioactivity, in vitro and in vivo, and high affinity binding to Flt-1 but not to Flk-1/KDR. J. Biol. Chem. 269(41), 25646-25654). Transfectants were selected and analyzed as described above for FAPα expression. The parental 293 cell line showed no detectable FAPα expression. One clone, referred to hereafter as 293-I/2, was FAPα positive.

### EXAMPLE 42

Examination of FAPα Expression in Transfected Cell Lines

FAPα expression was examined in the HT1080 and HT1080 clone 33 cells. Metabolic labeling, immunoprecipitations and fluorography were performed essentially as described (Park et al. (1991) Somatic Cell Mol Genet. 17(2), 137-150). HT1080 and HT1080 clone 33 cells were metabolically labelled with ³⁵S-methionine. Detergent extracts of these cells were immunoprecipitated with monoclonal antibody F 19 or with mouse IgG1 antibody as a negative control. Precipitates were boiled in sample buffer and separated by sodium dodecyl sulfate gel electrophoresis (as described by Laemmli (1970) Nature 227(259), 680-685). Fluorographic analysis of the resulting gel confirmed that the HT1080 clone 33 cells produce FAPα protein. No FAPα protein was detectable in extracts of the parental HT1080 cells nor in immunoprecipitates with mouse IgG1.

### EXAMPLE 43

Soluble Recombinant FAPα

A soluble recombinant form of FAPα protein was prepared as follows. A cDNA encoding the extracellular domain (ECD) of murine CD8a (Genbank M12825), consisting of the N-terminal 189 amino acids of CD8α, was ligated to a cDNA encoding the extracellular domain of FAPα (amino acids 27 to 760), generating a fusion protein construct, FAPmCD8, similar in structure to the CD8α-CD40 ligand fusion protein, as previously described (Lane et al. (1993) J. Exp. Med. 177(4), 1209-1213). The cDNAs were verified by sequencing and inserted into the pVL1393 vector. Transfection of Sf9 cells and amplification of the resulting recombinant baculovirus were performed as described (O'Reilly (1994) Baculovirus Expression Vectors: A Laboratory Manual, Oxford University Press, New York). The culture supernatant of High Five cells infected with recombinant FAPmCD8 baculovirus for four days was collected and cleared by ultracentrifugation. FAPmCD8 fusion protein was purified from such supernatants using an anti-FAPα monoclonal antibody immobilized on activated agarose beads (Pierce Chemical, Indianapolis, Ind., USA). The culture supernatant was passed through the antibody affinity column and eluted by pH shift using 0.1 M citrate buffer, pH 3. The samples were immediately neutralized with a saturated Tris solution (Sigma Chemicals, St. Louis, Mo.) and protein-containing fractions were pooled.

### EXAMPLE 44

Measurement of Cleavage of Doxorubicin-Peptide Conjugates

Samples were separated by reversed-phase high performance liquid chromatographic (HPLC) assay that was established to measure cleavage of doxorubicin-peptide conjugates. The HPLC system consisted of a Waters 717 autosampler equipped with a 1100 microliter (μl) loop and two Waters model 510 pumps to deliver solvents. Separations were performed under isocratic conditions at a flow rate of 0.7 ml/min on a Nucleosil C-18 column, 100 mm long×4 mm I.D. with 5 μm particle size (Dr. 1 ng. H. Knauer GmbH, Berlin). The mobile phase consisted of methanol:water (70:30, v/v) containing 0.2 M ammonium acetate, adjusted to pH 3.2. Free doxorubicin and doxorubicin-peptide conjugates were detected by fluorescence (excitation, 475 nm; emission, 585 nm) using a Waters 474 fluorescence detector. Injection, solvent delivery, data acquisition, and data analysis were all performed using the Millennium 2010 chromatography software package (Waters Corp., Milford, Mass., USA). Substances to be tested were first dissolved in dimethyl sulfoxide at a concentration of 5 mM and subsequently diluted in aqueous solution before being applied to the HPLC column.

The ability of soluble recombinant FAPα enzyme to release free doxorubicin from doxorubicin-peptide conjugates was examined. Doxorubicin-peptide conjugate stock solutions (5 mM) were diluted with Hepes-buffered saline pH 7.4 to a final concentration of 50 to 100 μM. Twenty μl of the resulting solution was mixed with 50 μl of purified FAPmCD8 fusion protein (approximately 20 ng) described above and 30 μl Hepes-buffered saline, pH 7.4. The mixture was allowed to incubate at 37° C. for 1 day and release of free doxorubicin was measured in the HPLC assay described. Areas under each peak were quantified using the software package above and the initial value was set to 100%. The rate of release of free doxorubicin was measured by the appearance of a peak with the same retention time as free doxorubicin under these HPLC conditions. The areas under each peak were used to calculate the relative amounts of free doxorubicin to doxorubicin-peptide conjugate. Integration of peak areas to determine percent cleavage was carried out using the Millennium 2010 chromatography software package above. The doxorubicin-peptide conjugate could be converted to free doxorubicin after incubation with purified FAPmCD8 fusion protein but the retention time of the conjugate was not altered by incubation with buffer.

### EXAMPLE 45

Reduction of Cytotoxicity of Doxorubicin by Conjugation to FAPα-Cleavable Peptides

The ability of FAPα-cleavable peptides to block the cytotoxic action of doxorubicin on FAPα-negative, doxorubicin-sensitive cells was determined. K562 cells, available from American Type Tissue Culture Collection, Rockville, Md., USA (ATCC Number: CCL-243), were seeded in 96 well plates (Greiner Scientific) at a density of 1000 cells/well. Serum-free cell culture media containing various concentrations of free doxorubicin or equivalent molar concentrations of doxorubicin-peptide conjugates were added to the cells.

Four days later, cell number was determined using an automated CASY™ cell counter (Schärfe System GmbH, Reutlingen, Germany).

### EXAMPLE 46

Release of Free Doxorubicin by Cell-Bound FAPα

The ability of cell-bound FAPα enzyme to release free doxorubicin from doxorubicin-peptide conjugates was examined. Each conjugate was dissolved in serum-free cell culture medium at a final concentration of 1 μM. Ten milliliters of this solution was added to confluent monolayers of HT1080 or HT1080 clone 33 cells in 10 cm tissue culture dishes for 19 hours at 37° C. The media were removed and release of doxorubicin measured as described in Example 44. The FAP-expressing cell line, HT1080 clone 33, converted the conjugates of examples 11 to 57 to free doxorubicin in high percentages.

### EXAMPLE 47

Killing of Sensitive Cells by FAPα-Released Doxorubicin

The ability of FAPα to generate free doxorubicin capable of killing doxorubicin-sensitive cells was determined. K562 cells, available from American Type Tissue Culture Collection, Rockville, Md., USA (ATCC Number: CCL-243), were seeded in 96 well plates (Greiner Scientific) at a density of 1000 cells/well. Serum-free cell culture media containing 1 μM doxorubicin-peptide conjugate was added to HT1080 or HT1080 clone 33 cells dishes for 19 hours at 37° C. The media were removed and release of doxorubicin was confirmed as in Example 44. Sixty-six μl of this medium was then added per well to the K562 cells. Four days later, cell number was determined using an automated CASY™ cell counter.

### EXAMPLE 48

Plasma Stability of Doxorubicin-Peptide Conjugates

The plasma stability of doxorubicin-peptide conjugates was measured using methods described in Example 61. Samples containing doxorubicin-peptide conjugates (at a concentration of 1 μM) were incubated in the presence of 10% (v/v) mouse or human plasma for the times indicated at 37° C.

FAPα-Catalyzed Cleavage of Selected 4-methoxy-β-napthylamide-peptide Conjugates

To identify preferred FAPα peptide substrates, blocked oligopeptides composed of natural amino carboxylic acids were synthesized and coupled to Proline-4-methoxy-β-napthylamine (Pro-MNA) using methods known to the art (E. Wünsch, Synthese von Peptiden, in Methoden der organischen Chemie, Houben-Weyl (Eds. E. Müller, 0. Bayer), Vol. XV, Part 1 and 2, Georg Thieme Verlag, Stuttgart, 1974). The turnover and cleavage rate have been determined with the aid pf the following assay:

Assay for Cleavage of MNA Substrates by FAP:

Buffer A:

100 mM Tris HCl pH 7.8, 100 mM NaCl

Cell extract from 293 cells stably transfected with FAP prepared as described (see Park, et al., Fibroblast Activation Protein, a Dual Specificity Serine Protease expressed in human tumor stromal fibroblasts. (1999) J. Biol. Chem. 36505-12.). A similar extract was also prepared from parental 293 control cells without FAP. The FAP concentration in the FAP-transfected cell extract was estimated by immunoassay and 1 ng enzyme (diluted in buffer A) was used per assay. FAP-negative 293 control cell extract was used at the same dilution (also in buffer A) as a negative control. Substrate was initially dissolved in dimethylformamide at a concentration of 200 mM and diluted in buffer A to a final concentration of 2.5 mM. A few substrates were not soluble at this concentration and had to be diluted further.

Assay Conditions:

10 μl 10% DMSO in buffer A

70 μl diluted FAP cell extract containing 1 ng FAP enzyme (OR control 293 cell extract without FAP)

20 μl 2.5 mM substrate

Mix, incubate at room temperature for 1 hour, and measure fluorescence in Fluorostar fluorimeter at the following wavelengths:

MNA conjugates: Excitation: 355 nm, Emission: 405 nm.

The fluorescence measured in the samples treated with control 293 control cell extracts without FAP is subtracted from the values measured in the samples treated with 1 ng FAP enzyme.

### EXAMPLE 49

Synthesis of 3,5-Difluorophenylacetyl-Gly-OH

H-Gly-Wang-resin (200 mg, 0.158) was added to a reaction vessel and washed with DMF (three times with 7 ml). 3,5-Difluorophenylacetic acid (163.2 mg, 0.95 mmol), 1-hydroxybenzotriazole (HOBt) (128.1, 0.95 mmol), diisopropylcarbodiimide (DIC) (146.8 μl, 0.95 mmol) and N,N dimethylformamide (DMF) (6 ml) were added to the reaction vessel. After 12 h of agitation, the resin was washed with DMF (six times with 7 ml), dichloromethane (DCM) (six times with 7 ml), methanol (MeOH) (six times with 7 ml), and Et₂O (six times with 7 ml) and treated with a solution of trifluoroacetic acid/water 95:5 (6 ml). After incubation for 2 h, the cleavage solution was placed into a flask and the resin was washed additionally with DCM (twice with 3 ml).

The cleavage solution was removed with a roto-evaporator and the resulting oil was dried with a stream of nitrogen. The crude product was purified by preparative reversed phase HPLC applying a acetonitrile/water gradient. The product gave satisfactory analytical data. HPLC>95%; ES-MS: m/z 219.2 ([M+H]⁺)

### EXAMPLES 50 TO 54

The following N-terminal blocked glycine amino acids were prepared analogously to this method:

| Example | Compound |
|---|---|
| 50 | 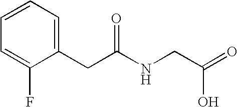 |
| 51 | 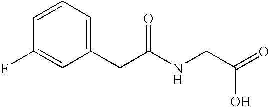 |
| 52 | 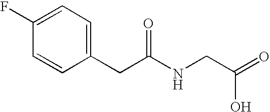 |
| 53 | 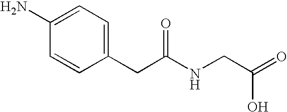 |
| 54 | 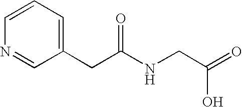 |

### EXAMPLE 55

Synthesis of Pyridin-3-ylmethoxycarbonyl-Pro-Ala-Gly-OH

H-Gly-Wang-resin (200 mg, 0.158) was added to a reaction vessel and washed with DMF (three times with 7 ml). Fmoc-Ala-OH (295.7 mg, 0.95 mmol), HOBt (128.1, 0.95 mmol), DIC (146.8 μl, 0.95 mmol) and DMF (6 ml) were added to the reaction vessel. After 6 h of agitation, the resin was then filtered, washed with DMF (eight times with 7 ml) and 20% piperidine in DMF (4 ml) was added to the reaction vessel. After agitation for 30 min, the resin was filtered and washed with DMF (eight times with 7 ml). Fmoc-Pro-OH was incorporated in the same manner. The Fmoc-group of proline was removed and the resin was washed with DMF (eight times with 7 ml) and DCM (six times with 7 ml). 3-Pyridylcarbinol (99 μl, 0.25 mmol) was treated with 4-nitrophenyl chloroformate (156 mg, 0.20 mmol) and triethylamine (225 μl, 0.4 mmol) in DCM (7 ml) for 6 h and the resulting mixture was added to the resin. After 12 h of agitation, the resin was washed with DCM (six times with 7 ml), MeOH) (six times with 7 ml), and Et₂O (six times with 7 ml) and treated with a solution of trifluoroacetic acid/water 95:5 (6 ml). After incubation for 2 h, the cleavage solution was placed into a flask and the resin was washed additionally with DCM (twice with 3 ml).

The cleavage solution was removed with a roto-evaporator and the resulting oil was dried with a stream of nitrogen. The crude product was purified by preparative reversed phase HPLC applying a acetonitrile/water gradient. The product gave satisfactory analytical data. HPLC>95%; ES-MS: m/z=378.4 ([M+H]⁺)

### EXAMPLE 56

Synthesis of Pyridin-3-ylmethoxycarbonyl-Pro-Ala-Gly-Pro-MNA

Pyridin-3-ylmethoxycarbonyl-Pro-Ala-Gly-OH (51.8 mg, 0,11 mmol), H-Pro-MNA (33.4 mg, 0.11 mmol), O-(benzotriazo-1-yl)-N,N,N′,N′,-tetramethyluronium-hexafluorophosphate (TBTU) (34.9 mg, 0.13 mmol), HOBt (14.7 mg, 0.11 mmol) and N-ethyldiisopropylamine (DIEA) (55.8 ml, 0.11 mmol) were dissolved in anhydrous DMF and stirred at 25° C. for 12 h. The solvent was then removed with a roto evaporator and the product was dissolved in DCM (10 ml). The DCM solution was washed with saturated aqueous sodium bicarbonate (30 ml) and brine (30 ml). The organic extract was dried with anhydrous MgSO₄, and the solvent was removed with a roto-evaporator. The product was chromatographed by preparative RP-HPLC on C18 using a gradient of water/acetonitrile with 0.1% trifluoroacetic acid.

HPLC>95%; ES-MS: m/z 631 ([M+H]⁺)

The following table shows the peptide-MNA-conjugates which have been prepared analogously and includes cleavage data by FAP.

| | | Cleavage |
|---|---|---|
| MNA-Conjugate | % Turnover | [μM] |
| 4-Amino-Phenylacetyl-Gly-Pro-MNA | 0.6 | 3 |
| 3,5-Difluorphenylacetyl-Gly-Pro-MNA | 2.26 | 2.26 |
| 2-Fluorphenylacetyl-Gly-Pro-MNA | 0.26 | 1.28 |
| 3-Fluorphenylacetyl-Gly-Pro-MNA | 0.35 | 1.77 |
| 4-Fluorphenylacetyl-Gly-Pro-MNA | 0.42 | 2.08 |
| 3-Pyridylacetyl-Gly-Pro-MNA | 1.82 | 9.12 |
| 3 -Pyridylmethyloxycarbonyl-Gly-Pro-MNA | 0.72 | 3.58 |
| 4-Pyridylmethyloxycarbonyl-Gly-Pro-MNA | 1.18 | 5.92 |
| 3-Pyridylmethyloxycarbonyl-Pro-Ala-Gly-Pro- | 1.77 | 8.86 |
| MNA | | |
| 4-Pyridylmethyloxycarbonyl-Pro-Ala-Gly-Pro- | 1.28 | 6.38 |
| MNA | | |
| 4-Aminomethylbenzoyl-Pro-Ala-Gly-Pro-MNA | 0.24 | 1.21 |
| 4-Aminomethylphenylacetyl-Pro-Ala-Gly-Pro- | 0.26 | 1.28 |
| MNA | | |
| 4-(2-Aminothiazol-5-yl)-acetyl-Pro-Ala-Gly- | 0.39 | 1.94 |
| Pro-MNA | | |
| 4-Hydroxyphenylacetyl-Pro-Ala-Gly-Pro-MNA | 0.69 | 1.72 |
| Succinyl-Pro-Ala-Gly-Pro-MNA | 0.29 | 1.44 |
| Glutaryl-Pro-Ala-Gly-Pro-MNA | 0.3 | 1.48 |
| 2-Morpholino-ethyl-Pro-Ala-Gly-Pro-MNA | 0.26 | 0.64 |
| Morpholino-carbonyl-Pro-Ala-Gly-Pro-MNA | 0.6 | 2.99 |
| Morpholino-(ethyloxycarbonyl)-Pro-Ala-Gly- | 0.65 | 3.25 |
| Pro-MNA | | |
| 4-Dimethylaminobenzoyl-Pro-Ala-Gly-Pro- | 0.37 | 1.85 |
| MNA | | |
| Prazin-2-ylcarbonyl-Pro-Ala-Gly-Pro-MNA | 0.56 | 0.7 |
| 1-Methylpyrrol-2-ylcarbonyl Pro-Ala-Gly- | 0.13 | 0.33 |
| Pro-MNA | | |
| Malonyl-Pro-Ala-Gly-Pro-MNA | 0.21 | 1.05 |
| 3-Carboxyphenylsulfonyl-Pro-Ala-Gly-Pro- | 0.58 | 2.88 |
| MNA | | |
| Pyrid-4-ylmethyloxycarbonyl-Pip-Ala-Gly- | 0.43 | 2.16 |
| Pro-MNA¹ | | |
| Pyrid-4-ylmethyloxycarbonyl-2-Aze-Ala-Gly- | 1.61 | 8.07 |
| Pro-MNA² | | |
| Pyrid-3-ylmethyloxycarbonyl-Pip-Ala-Gly-Pro- | 0.56 | 2.78 |
| MNA¹ | | |
| Pyrid-3-ylmethyloxycarbonyl-MeAla-Ala-Gly- | 1.27 | 6.31 |
| Pro-MNA³ | | |
| Pyrid-3-ylmethyloxycarbonyl-2-Aze-Ala-Gly- | 3.42 | 17.1 |
| Pro-MNA² | | |

¹Pip represents a piperidin-2-ylcarboxylic acid moiety
²2-Aze represents an azetidin-2-ylcarboxylic acid moiety
³MeAla represents a N-methylalanin moiety

### EXAMPLE 57

Preparation of Pyridin-3-ylmethoxycarbonyl-Pro-Ala-Gly-Pro-Melphalan

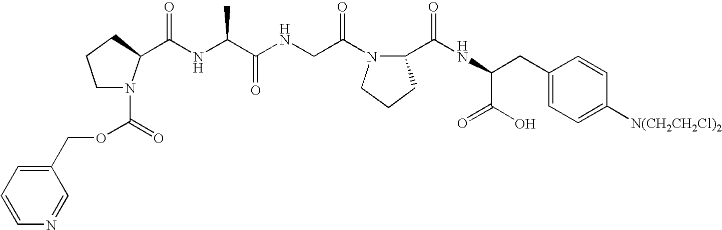

Pyridin-3-ylmethoxycarbonyl-Pro-Ala-Gly-Pro-OH (0.072 mmol) was dissolved in anhydrous N,N-dimethylformamide (8 ml) and pH was adjusted to 7.5 by N,N-diisopropylethylamine. N-hydroxysuccinimide (0.072 mmol) was added and the mixture was cooled in an ice bath. Under stirring, dicyclohexylcarbodiimide (0.67 mmol) was added and the solution was stirred at 0° C. for 2 h.

Melphalan (0.048 mmol) was dissolved in 30 ml anhydrous DMF and N,N-diisopropylethylamine (0.072 mmol) was added. This mixture was syringed to the activated peptide. The reaction was allowed to warm up to room temperature and was stirred for 24 h. The solvent was then removed and the product was purified by preparative RP-HPLC on C18 using a gradient of water/acetonitrile with 0.1% trifluoracetic acid.

1. A compound of formula (I) 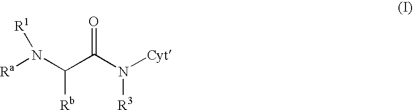
or a pharmaceutically acceptable salt thereof,
wherein
R¹ represents an amino alkanoyl or oligopeptidoyl group, the N-terminal amino function of which is attached to a capping group (Cg) which is capable of enhancing the chemical stability of said compound under physiological conditions and the physical stability of aqueous pharmaceutical formulations comprising said compound;
R^{a} and R^{b} together with the interjacent N—C group form an optionally substituted, optionally benzo- or cyclohexano-condensed 3- to 7-membered saturated or unsaturated heterocyclic ring, in which one or two CH₂ groups may also be replaced by NH, O or S;
R³ represents H, C₁-C₆-alkyl, C₃-C₈-cycloalkyl, aryl or heteroaryl; and
Cyt′ represents the residue of a cytotoxic or cytostatic compound:

2. A compound of formula (I) according to claim 1 wherein the capping group exhibits one or more functional groups, which have the capability of forming salts with pharmaceutially acceptable acids or bases, selected from amino, carboxy, phospate, phophonate, sulfate and sulfonate groups.

3. A compound of formula (I) according to claim 1 or 2, wherein said capping group (Cg) is a group of formula
R²—(CH₂)ₘ-Z-,
in which
R² represents
(f) a group selected from C₁-C₆ alkyl, C₃-C₈ cycloalkyl, aryl and heteroaryl, wherein each of these groups is substituted by at least one amino, carboxy, phospate, phophonate, sulfate, sulfonate or hydroxy group, or
(g) an optionally substituted 5- to 7-membered saturated or unsaturated nitrogen, oxygen and/or sulfur containing heterocyclic group,
(h) a phenyl group which is substituted by 1 to 5 fluorine atoms;
(i) a C₁-C₆ fluoroalkyl group; or
(j) in the case that m is 1, an optionally substituted 5- to 6-membered heteroaryl group;
Z represents —CO—, —O—CO—, —SO₂—, NH—CO— or a single bond;
m is 0, 1 or 2.

4. A compound of formula (I) according to claim 1 or 2, wherein
R¹ represents a residue of formula Cg-A, Cg-B-A or Cg-(D)ₙ-B-A, in which
Cg represents a capping group of formula R²—(CH₂)ₘ-Z-, wherein R² is an optionally substituted saturated heterocyclyl or heteroaryl group; m is 0 or 1;
A, B and D each independently represent moieties derived from amino carboxylic acids of the formula —[NR⁴—(X)ₚ—CO]— wherein X represents CR⁵R⁶ and wherein R⁴, R⁵ and R⁶ each independently represent a hydrogen atom, an optionally substituted C₁-C₆-alkyl, C₃-C₈-cycloalkyl, aryl, aralkyl, heteroaryl or heteroarylalkyl group, and
p is 1, 2, 3, 4, 5; or
A, B and D each independently represent moieties derived from cyclic amino carboxylic acids of formula 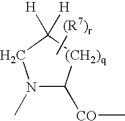
wherein
R⁷ represents C₁-C₆-alkyl, OH, or NH₂,
n is an integer from 1 to 10;
q is 0, 1 or 2; and
r is 0, 1 or 2.

5. A compound of formula I according to any of claims 1 to 3, wherein the heterocyclic ring formed by R^{a}, R^{b} and the interjacent N—C is substituted by R⁸ and R⁹, wherein R⁸ and R⁹ each independently represent a hydrogen or halogen atom or a C₁-C₆-alkyl, C₁-C₆-alkylamino, di-C₁-C₆-alkylamino, C₁-C₆-alkoxy, thiol, C₁-C₆-alkylthio, oxo, imino, fomyl, C₁-C₆-alkoxy carbonyl, amino carbonyl, C₃-C₈-cycloalkyl, aryl, or heteroaryl group.

6. A compound of fomula IA 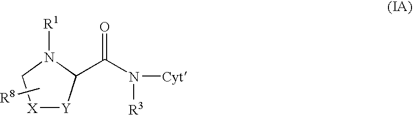
wherein R¹, R³, R⁸, Cyt′ are as defined in any of the preceding claims, and
X-Y represents CHR⁹—CH₂, CR²═CH, NH—CH₂, CH₂—NH, —CR⁹—, CH₂—CHR⁹—CH₂.

7. A compound of fomula IA1 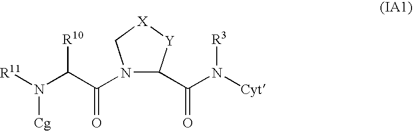
wherein R³, Cyt′ Cg, X and Y are as defined in any of the preceding claims, and R¹⁰ and R¹¹ each independently represent a hydrogen atom, an optionally substituted C₁-C₆-alkyl, C₃-C₈-cycloalkyl, aryl or heteroaryl group, or
R¹⁰ and R¹¹ together with the interjacent N—C group form an optionally substituted, optionally benzo- or cyclohexano-condensed 3- to 7-membered saturated or unsaturated heterocyclic ring, in which one or two CH₂ groups may also be replaced by NH, O or S.

8. A compound of fomula IA2 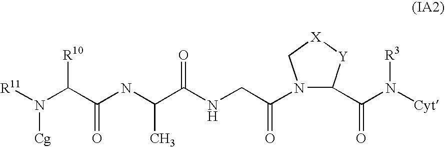
wherein R³, Cyt′ Cg, X and Y are as defined in any of the preceding claims, and R¹⁰ and R¹¹ each independently represent a hydrogen atom, an optionally substituted C₁-C₆-alkyl, C₃-C₈-cycloalkyl, aryl or heteroaryl group or
R¹⁰ and R¹¹ together with the interjacent N—C group form an optionally substituted, optionally benzo- or cyclohexano-condensed 3- to 7-membered saturated or unsaturated heterocyclic ring.

9. Compounds of formulae I, IA, IA1 or IA2 according to any of the preceding claims, wherein
R² represents a group selected from

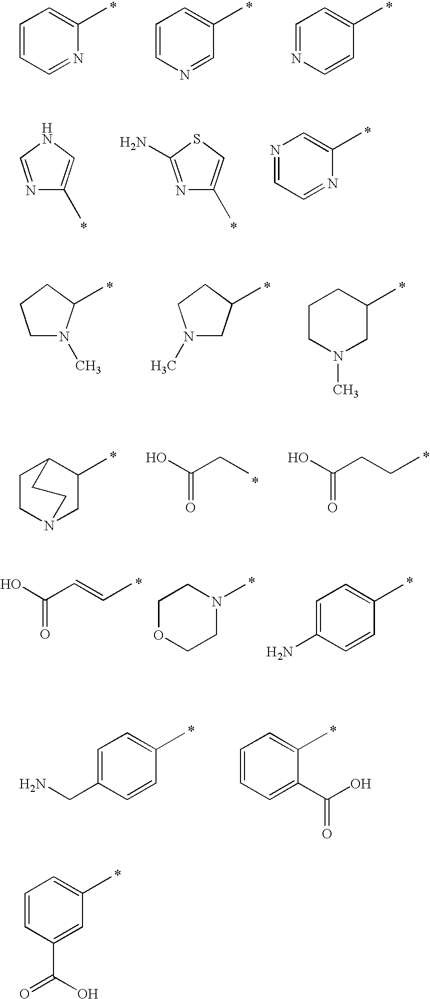

10. A compound according to any of the preceding claim, wherein R¹ represents an aminoalkanoyl, or an oligopeptidoyl group, which is derived from glycine (Gly), or the D- or L-forms of alanine (Ala), valine (Val), leucine (Leu), isoleucine (Ile), phenylalanine (Phe), tyrosine (Tyr), tryptophan (Trp), cysteine (Cys), methionine (Met), serine (Ser), threonine (Thr), lysine (Lys), arginine (Arg), histidine (His), aspartatic acid (Asp), glutamic acid (Glu), asparagine (Asn), glutamine (Gln), proline (Pro), 4-hydroxy-proline (Hyp), 5-hydroxy-lysine, norleucine (Nle), 5-hydroxynorleucine (Hyn), 6-hydroxynorleucine, ornithine, or cyclohexylglycine (Chg) and wherein the N-terminal amino function of said aminoalkanoyl or oligopeptidoyl group is attached to a capping group Cg.

11. A compound of formula I according to any of the preceding claims, wherein the unit A is derived from L-proline, glycine, L-norleucine, L-cyclohexylglycine, L-5-hydroxynorleucine, L-6-hydroxynorleucine, L-5-hydroxylysine, L-arginine, or L-lysine.

12. A compound according to any of the preceding claims wherein R¹ is a group selected from the formulae (1) to (14):
Cg-Gly (1)
Cg-Nle (2)
Cg-Val (3)
Cg-Met (4)
Cg-Xxx-Gly (5)
Cg-Xxx-Hyn (6)
Cg-Xxx-Pro (7)
Cg-Xxx-His (8)
Cg-Xxx-Met (9)
Cg-Xxx-Ala (10)
Cg-Xxx-Hyn (11)
Cg-Xxx-Ala-Gly (12)
Cg-(Xxx)ₙ—Xxx-Gly (13)
Cg-(Xxx)ₙ—Xxx-Ala-Gly (14)
wherein
Cg represents a capping group selected from pyridinyloxycarbonyl, pyridinylacetyl, pyridinylmethylsulfonyl and pyridylmethylaminocarbonyl;
Xxx represents a moiety derived from an amino carboxylic acid; and
n is an integer from 1 to 6.

13. A compound according to claim 11 wherein the amino alkanoic acid moieties exist in the (L)-configuration

14. A compound of any one of claims 1 to 12, wherein Cyt′ is an anthracycline group.

15. A compound of claim 14 selected from the formulae (IIIA) to (IIIH):

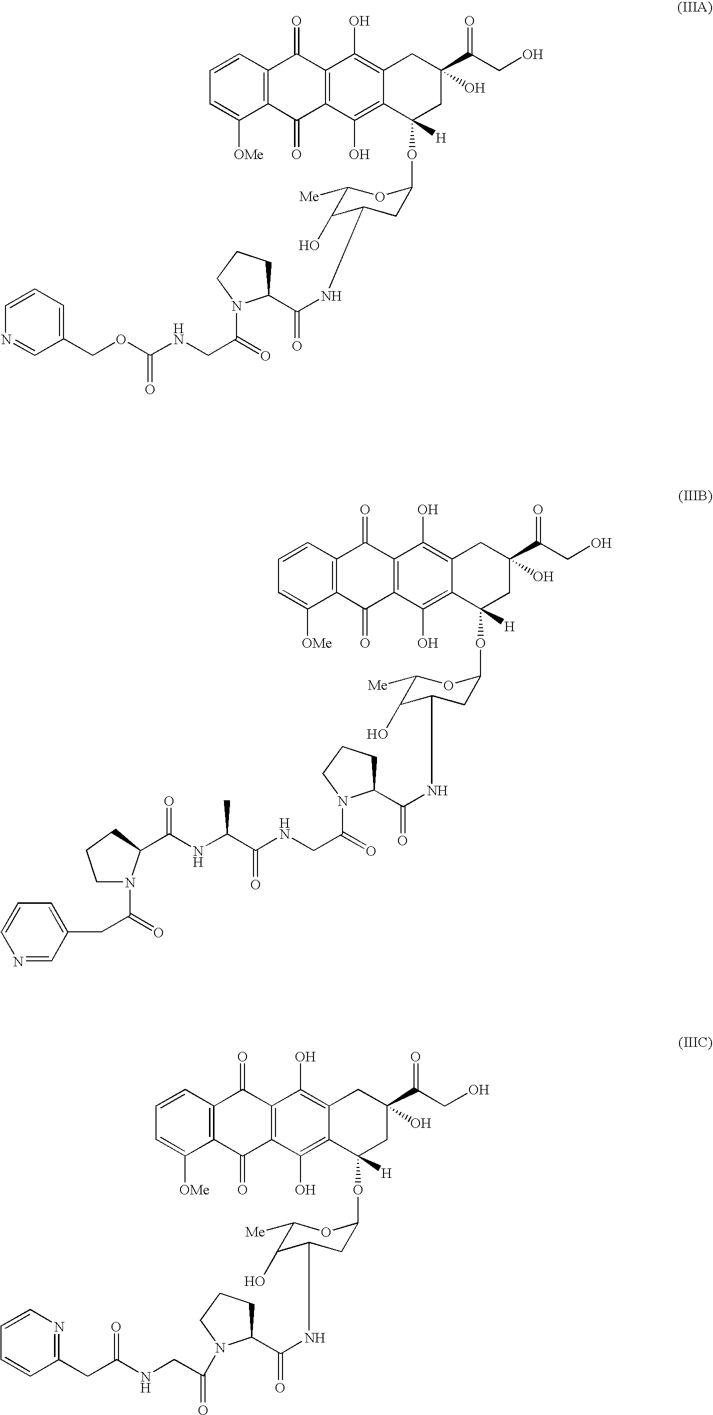

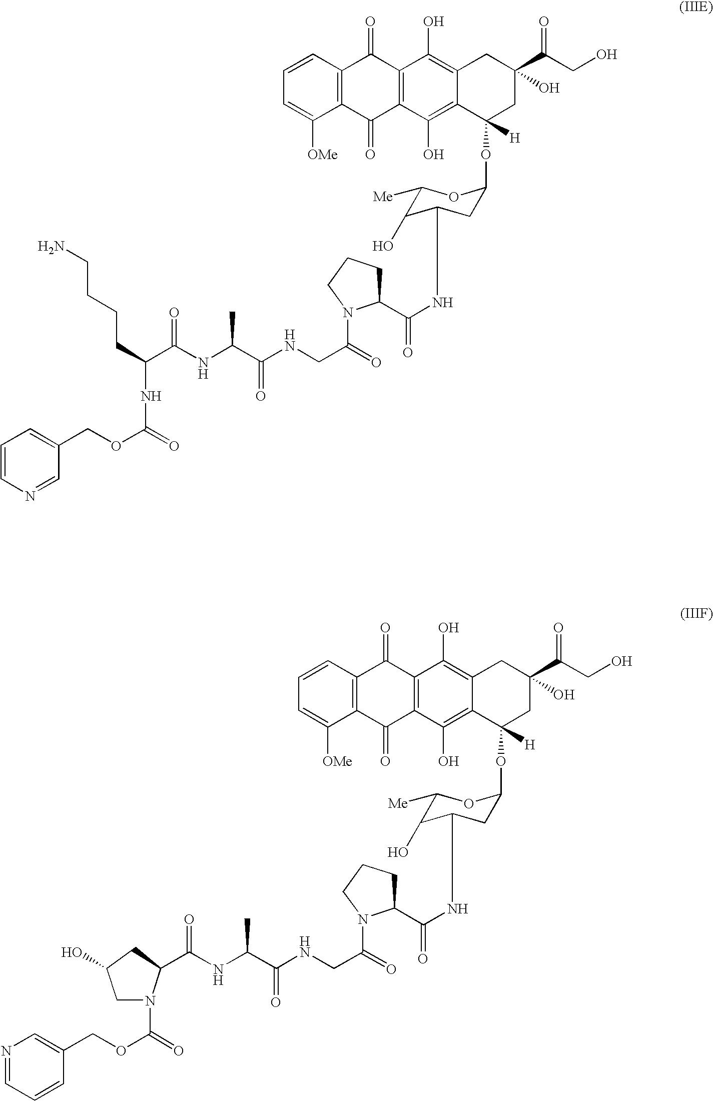

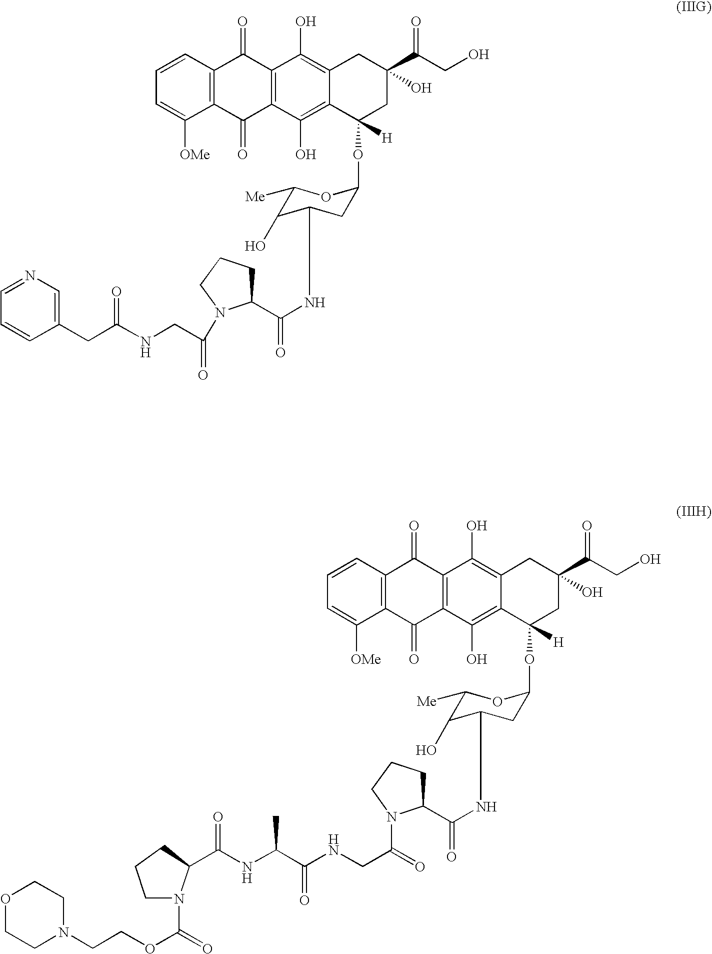

16. A prodrug that is capable of being converted into a cytotoxic or cytostatic drug, by the catalytic action of FAPα, said prodrug exhibits an oligomeric part comprising up to 13 amino carboxylic residues, the C-terminal amino carboxylic thereof is recognised by FAPα, and a cytotoxic or cytostatic part, characterized in that
the N-terminal amino function of the oligomeric part is attached to a capping group (Cg) which is capable of enhancing the chemical stability of said prodrug under physiological conditions and the physical stability of an aqueous pharmaceutical formulations comprising said prodrug.

17. The prodrug of claim 16 wherein the capping group is a group of formula R²—(CH₂)ₘ-Z-, in which R² represents
(a) a group selected from C₁-C₆ alkyl, C₃-C₈ cycloalkyl, aryl and heteroaryl, wherein each of these groups is substituted by at least one amino, carboxy or hydroxy group, or
(b) an optionally substituted 5- to 7-membered saturated, unsaturated or aromatic nitrogen containing heterocyclic group, or
(c) a phenyl group which is substituted by 1 to 5 fluorine atoms;
Z represents —CO—, —O—CO—, —SO₂—, —NH—CO— or a single bond;
m is 0 or 1.

18. The prodrug of claim 16 or 17, wherein the C-terminal amino carboxilic residue is selected from D-proline, L-proline, D-hydroxyproline and L-hydroxyproline and the oligomeric part comprises two, three, or four amino carboxylic acid residues.

19. A compound of any one of the preceding claims for medical use.

20. Pharmaceutical composition comprising a compound according to any one of claims 1 to 19, and optionally one or more pharmaceutically acceptable excipients.

21. Use of a compound according to any one of claims 1 to 19 in the preparation of a pharmaceutical composition for the treatment of cancer.

22. Method of treatment of cancer, comprising administering a pharmaceutical composition according to claim 20 to a patient.

23. Method of treatment of cancer, wherein a prodrug is administered to a patient wherein said prodrug is capable of being converted into a cytotoxic or cytostatic drug by the enzymatic activity of FAPα, and said prodrug exhibits an oligomeric part comprising up to 13 amino carboxylic residues, the C-terminal amino carboxylic thereof is recognised by FAPα, and a cytotoxic or cytostatic part, characterized in that the N-terminal amino function of the oligomeric part is attached to a capping group (Cg) which is capable of enhancing the chemical stability of said prodrug under physiological conditions and the physical stability of aqueous pharmaceutical formulations comprising said prodrug.

24. Use of a prodrug which is capable of being converted into a cytotoxic or cytostatic drug by the enzymatic activity of FAPα, said prodrug exhibits an oligomeric part comprising up to 13 amino carboxylic residues, the C-terminal amino carboxylic thereof is recognised by FAPα, and a cytotoxic or cytostatic part, wherein the N-terminal amino function of the oligomeric part is attached to a capping group (Cg) which is capable of enhancing the chemical stability of said prodrug under physiological conditions and the physical stability of aqueous pharmaceutical formulations comprising said prodrug, for the manufacture of a stable medicament for the treatment of cancer.
